SEQUENCE LISTING (32 sequences)

SEQ ID NO: 1 - DNA, 1719 bp, Human Nogo receptor-1
agcccagccagagccgggcggagcggagcgcgccgagcctcgtcccgcggccgggccggggccgggccgtagcggcggcgcctggatgcggacccggccgcggggagacgggcgcccgccccgaaacgactttcagtccccgacgcgccccgcccaacccctacgatgaagagggcgtccgctggagggagccggctgctggcatgggtgctgtggctgcaggcctggcaggtggcagccccatgcccaggtgcctgcgtatgctacaatgagcccaaggtgacgacaagctgcccccagcagggcctgcaggctgtgcccgtgggcatccctgctgccagccagcgcatcttcctgcacggcaaccgcatctcgcatgtgccagctgccagcttccgtgcctgccgcaacctcaccatcctgtggctgcactcgaatgtgctggcccgaattgatgcggctgccttcactggcctggccctcctggagcagctggacctcagcgataatgcacagctccggtctgtggaccctgccacattccacggcctgggccgcctacacacgctgcacctggaccgctgcggcctgcaggagctgggcccggggctgttccgcggcctggctgccctgcagtacctctacctgcaggacaacgcgctgcaggcactgcctgatgacaccttccgcgacctgggcaacctcacacacctcttcctgcacggcaaccgcatctccagcgtgcccgagcgcgccttccgtgggctgcacagcctcgaccgtctcctactgcaccagaaccgcgtggcccatgtgcacccgcatgccttccgtgaccttggccgcctcatgacactctatctgtttgccaacaatctatcagcgctgcccactgaggccctggcccccctgcgtgccctgcagtacctgaggctcaacgacaacccctgggtgtgtgactgccgggcacgcccactctgggcctggctgcagaagttccgcggctcctcctccgaggtgccctgcagcctcccgcaacgcctggctggccgtgacctcaaacgcctagctgccaatgacctgcagggctgcgctgtggccaccggcccttaccatcccatctggaccggcagggccaccgatgaggagccgctggggcttcccaagtgctgccagccagatgccgctgacaaggcctcagtactggagcctggaagaccagcttcggcaggcaatgcgctgaagggacgcgtgccgcccggtgacagcccgccgggcaacggctctggcccacggcacatcaatgactcaccctttgggactctgcctggctctgctgagcccccgctcactgcagtgcggcccgagggctccgagccaccagggttccccacctcgggccctcgccggaggccaggctgttcacgcaagaaccgcacccgcagccactgccgtctgggccaggcaggcagcgggggtggcgggactggtgactcagaaggctcaggtgccctacccagcctcacctgcagcctcacccccctgggcctggcgctggtgctgtggacagtgcttgggccctgctgacccccagcggacacaagagcgtgctcagcagccaggtgtgtgtacatacggggtctctctccacgccgccaagccagccgggcggccgacccgtggggcaggccaggccaggtcctccctgatggacgcctg

SEQ ID NO: 2 - protein, 473 aa, Human NgR-1
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKASVLEPGRPASAGNALKGRVPPGDSPPGNGSGPRHINDSPFGTLPGSAEPPLTAVRPEGSEPPGFPTSGPRRRPGCSRKNRTRSHCRLGQAGSGGGGTGDSEGSGALPSLTCSLTPLGLALVLWTVLGPC

SEQ ID NO: 3 - protein, 15 aa, Artificial Sequence
  Synthetic “linker”
GGGGSGGGGSGGGGS

SEQ ID NO: 4 - protein, 15 aa, Artificial Sequence
  Synthetic “linker”
ESGRSGGGGSGGGGS

SEQ ID NO: 5 - protein, 14 aa, Artificial Sequence
  Synthetic “linker”
EGKSSGSGSESKST

SEQ ID NO: 6 - protein, 15 aa, Artificial Sequence
  Synthetic “linker”
EGKSSGSGSESKSTQ

SEQ ID NO: 7 - protein, 14 aa, Artificial Sequence
  Synthetic “linker”
EGKSSGSGSESKVD

SEQ ID NO: 8 - protein, 14 aa, Artificial Sequence
  Synthetic “linker”
GSTSGSGKSSEGKG

SEQ ID NO: 9 - protein, 18 aa, Artificial Sequence
  Synthetic “linker”
KESGSVSSEQLAQFRSLD

SEQ ID NO: 10 - protein, 16 aa, Artificial Sequence
  Synthetic “linker”
ESGSVSSEELAFRSLD

SEQ ID NO: 11 - protein, 8 aa, Artificial Sequence
  Synthetic epitope tag used to identify, purify, concentrate, or isolate the NgR polypeptide.
DYKDDDDK

SEQ ID NO: 12 - protein, 8 aa, Artificial Sequence
  Synthetic epitope tag
DYKDEDDK

SEQ ID NO: 13 - protein, 9 aa, Artificial Sequence
  Synthetic epitope tag
AWRHPQFGG

SEQ ID NO: 14 - protein, 11 aa, Artificial Sequence
  Synthetic epitope tag
YTDIEMNRLGK

SEQ ID NO: 15 - protein, 6 aa, Artificial Sequence
  Synthetic epitope tag
HHHHHH

SEQ ID NO: 16 - protein, 13 aa, Influenza Virus Hemagglutinin (HA)
YPYDVPDYAIEGR

SEQ ID NO: 17 - protein, 11 aa, Human c-myc (Myc)
EQKLLSEEDLN

SEQ ID NO: 18 - protein, 13 aa, Artificial Sequence
  Tryptic digest of NgR-1
LCPGACVCYNEPK

SEQ ID NO: 19 - protein, 6 aa, Artificial Sequence
  Tryptic digest of NgR-1
DNPWVC

SEQ ID NO: 20 - protein, 13 aa, Artificial Sequence
  Tryptic digest of NgR-1
GSSSEVPCSLPQR

SEQ ID NO: 21 - protein, 9 aa, Artificial Sequence
  Tryptic digest of NgR-1
CCQPDAADK

SEQ ID NO: 22 - protein, 412 aa, Human NgR-1
CPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKASVLEPGRPASAGNALKGRVPPGDSPPGNGSGPRHINDSPFGTLPGSAEPPLTAVRPEGSEPPGFPTSGPRRRPGCSRKNRTRSHCRLGQAGSGG

SEQ ID NO: 23 - protein, 473 aa, Rat NgR1
MKRASSGGSRLLAWVLWLQAWRVATPCPGACVCYNEPKVTTSCPQQGLQAVPTGIPASSQRIFLHGNRISHVPAASFQSCRNLTILWLHSNALARIDAAAFTGLTLLEQLDLSDNAQLHVVDPTTFHGLGHLHTLHLDRCGLRELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLMPLRSLQYLRLNDNPWVCDCRARPLWAWLQKFRGSSSEVPCNLPQRLADRDLKRLAASDLEGCAVASGPFRPIQTSQLTDEELLSLPKCCQPDAADKASVLEPGRPASAGNALKGRVPPGDTPPGNGSGPRHINDSPFGTLPSSAEPPLTALRPGGSEPPGLPTTGPRRRPGCSRKNRTRSHCRLGQAGSGASGTGDAEGSGALPALACSLAPLGLALVLWTVLGPC

SEQ ID NO: 24 - protein, 420 aa, Human NgR2
MLPGLRRLLQAPASACLLLMLLALPLAAPSCPMLCTCYSSPPTVSCQANNFSSVPLSLPPSTQRLFLQNNLIRTLRPGTFGSNLLTLWLFSNNLSTIYPGTFRHLQALEELDLGDNRHLRSLEPDTFQGLERLQSLHLYRCQLSSLPGNIFRGLVSLQYLYLQENSLLHLQDDLFADLANLSHLFLHGNRLRLLTEHVFRGLGSLDRLLLHGNRLQGVHRAAFRGLSRLTILYLFNNSLASLPGEALADLPSLEFLRLNANPWACDCRARPLWAWFQRARVSSSDVTCATPPERQGRDLRALREADFQACPPAAPTRPGSRARGNSSSNHLYGVAEAGAPPADPSTLYRDLPAEDSRGRQGGDAPTEDDYWGGYGGEDQRGEQMCPGAACQAPPDSRGPALSAGLPSPLLCLLLLVPHHL

SEQ ID NO: 25 - DNA, 1422 bp, Rat Nogo receptor-1
atgaagagggcgtcctccggaggaagccggctgccgacatgggtgttatggctacaggcctggagggtagcaacgccctgccctggtgcctgtgtgtgctacaatgagcccaaggtcacaacaagccgcccccagcagggcctgcaggctgtacccgctggcatcccagcctccagccagagaatcttcctgcacggcaaccgaatctcttacgtgccagccgccagcttccagtcatgccggaatctcaccatcctgtggctgcactcaaatgcgctggccgggattgatgccgcggccttcactggtctgaccctcctggagcaactagatcttagtgacaatgcacagctccgtgtcgtggaccccaccacgttccgtggcctgggccacctgcacacgctgcacctagaccgatgcggcctgcaggagctggggcctggcctattccgtgggctggcagctctgcagtacctctacctacaagacaacaacctgcaggcacttcccgacaacaccttccgagacctgggcaacctcacgcatctctttctgcatggcaaccgtatccccagtgttcctgagcacgctttccgtggcttgcacagtcttgaccgtctcctcttgcaccagaaccatgtggctcgtgtgcacccacatgccttccgggaccttggccgactcatgaccctctacctgtttgccaacaacctctccatgctccccgcagaggtcctagtgcccctgaggtctctgcagtacctgcgactcaatgacaacccctgggtgtgtgactgcagggcacgtccgctctgggcctggctgcagaagttccgaggttcctcatccggggtgcccagcaacctaccccaacgcctggcaggccgtgatctgaagcgcctggctaccagtgacttagagggttgtgctgtggcttcggggcccttccgtcccttccagaccaatcagctcactgatgaggagctgctgggcctccccaagtgctgccagccggatgctgcagacaaggcctcagtactggaacccgggaggccggcgtctgttggaaatgcactcaagggacgtgtgcctcccggtgacactccaccaggcaatggctcaggcccacggcacatcaatgactctccatttgggactttgcccggctctgcagagcccccactgactgccctgcggcctgggggttccgagcccccgggactgcccaccacgggcccccgcaggaggccaggttgttccagaaagaaccgcacccgtagccactgccgtctgggccaggcaggaagtgggagcagtggaactggggatgcagaaggttcgggggccctgcctgccctggcctgcagccttgctcctctgggccttgcactggtactttggacagtgcttgggccctgctga

SEQ ID NO: 26 - DNA, 1263 bp, Human Nogo receptor-2
atgctgcccgggctcaggcgcctgctgcaagctcccgcctcggcctgcctcctgctgatgctcctggccctgcccctggcggcccccagctgccccatgctctgcacctgctactcatccccgcccaccgtgagctgccaggccaacaacttctcctctgtgccgctgtccctgccacccagcactcagcgactcttcctgcagaacaacctcatccgcacgctgcggccaggcacctttgggtccaacctgctcaccctgtggctcttctccaacaacctctccaccatctacccgggcactttccgccacttgcaagccctggaggagctggacctcggtgacaaccggcacctgcgctcgctggagcccgacaccttccagggcctggagcggctgcagtcgctgcatttgtaccgctgccagctcagcagcctgcccggcaacatcttccgaggcctggtcagcctgcagtacctctacctccaggagaacagcctgctccacctacaggatgacttgttcgcggacctggccaacctgagccacctcttcctccacgggaaccgcctgcggctgctcacagagcacgtgtttcgcggcctgggcagcctggaccggctgctgctgcacgggaaccggctgcagggcgtgcaccgcgcggccttccgcggcctcagccgcctcaccatcctctacctgttcaacaacagcctggcctcgctgcccggcgaggcgctcgccgacctgccctcgctcgagttcctgcggctcaacgctaacccctgggcgtgcgactgccgcgcgcggccgctctgggcctggttccagcgcgcgcgcgtgtccagctccgacgtgacctgcgccacccccccggagcgccagggccgagacctgcgcgcgctccgcgaggccgacttccaggcgtgtccgcccgcggcacccacgcggccgggcagccgcgcccgcggcaacagctcctccaaccacctgtacggggtggccgaggccggggcgcccccagccgatccctccaccctctaccgagatctgcctgccgaagactcgcgggggcgccagggcggggacgcgcctactgaggacgactactgggggggctacgggggtgaggaccagcgaggggagcagatgtgccccggcgctgcctgccaggcgcccccggactcccgaggccctgcgctctcggccgggctccccagccctctgctttgcctcctgctcctggtgccccaccacctctga

SEQ ID NO: 27 - protein, 11 aa, Artificial Sequence
  Tryptic digest of NgR-1
LNDNPWVCDCR

SEQ ID NO: 28 - protein, 9 aa, Artificial Sequence
  Tryptic digest of NgR-1
CCQPDAADK

SEQ ID NO: 29 - protein, 23 aa, Artificial Sequence
  Tryptic digest of NgR-1
LAANDLQGCAVATGPYHPIWTGR

SEQ ID NO: 30 - protein, 13 aa, Artificial Sequence
  Tryptic digest of rat NgR-1
GSSSEVPCNLPQR

SEQ ID NO: 31 - protein, 11 aa, Artificial Sequence
  Tryptic digest of rat NgR-1
LNDNPWVCGCR

SEQ ID NO: 32 - protein, 10 aa, Artificial Sequence
  Tryptic digest of rat NgR-1
LAASDLQGCA

(19) United States
(12) Patent Application Publication
(10) Pub. No.: US 2009/0062199 A1
(43) Pub. Date: Mar. 5, 2009

(54) Nogo Receptor Polypeptides and Polypeptide Fragments and Uses Thereof

(75) Inventors: Dingyi Wen, Waltham, MA (US); Daniel H.S. Lee, Sudbury, MA (US); R. Blake Pepinsky, Arlington, MA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc., Cambridge, MA (US)

(21) Appl. No.: 12/064,753

(22) PCT Filed: Aug. 25, 2006
(86) PCT No.: PCT/US2006/033369

§ 371 Date: Sep. 18, 2008

Related U.S. Application Data
(60) Provisional application No. 60/710,864, filed on Aug. 25, 2005.

(51) Int. Cl. A61K 38/17; C07K 14/47; C12N 15/12; A61K 31/711; A61P 25/00; C12N 15/85; C12N 5/10

(52) U.S. Cl. 514/12; 530/300; 536/23.5; 435/320.1; 435/325; 514/44; 435/375

(57) ABSTRACT

Nogo receptor 1 (NgR1) is a leucine rich repeat protein that forms part of a signaling complex that modulates axon regeneration. Previous studies have shown that the entire LRR region of Nogo receptor-1, including the C-terminal cap of LRR, LRRCT, is needed for ligand binding, and that the adjacent CT stalk of the Nogo receptor-1 contributes to interaction with its co-receptors. The present invention is directed to the use of certain Nogo receptor-1 and Nogo receptor-2 polypeptides and polypeptide fragments for promoting neurite outgrowth, neuronal survival, and axonal regeneration in CNS neurons. The invention features molecules and methods useful for inhibiting neurite outgrowth inhibition, promoting neuronal survival, and/or promoting axonal regeneration in CNS neurons.

### BACKGROUND OF THE INVENTION

1. Field of the Invention

This invention relates to neurobiology, neurology and pharmacology. More particularly, the invention relates to neurons and compositions and methods for mediating axonal growth.

2. Background Art

Axons and dendrites of neurons are long cellular extensions from neurons. The distal tip of an extending axon or neurite comprises a specialized region known as the growth cone, which senses the local environment and guides axonal growth toward the neuron's target cell. The guidance of growth at the cone involves various classes of adhesion molecules, intercellular signals, as well as factors that stimulate and inhibit growth cones.

Nerve cell function is greatly influenced by the contact between the neuron and other cells in its immediate environment. These cells include specialized glial cells, oligodendrocytes in the central nervous system (CNS), and Schwann cells in the peripheral nervous system (PNS), which ensheathe the neuronal axon with myelin (an insulating structure of multi-layered membranes). While CNS neurons have the capacity to regenerate after injury, they are inhibited from doing so because of the presence of inhibitory proteins present in myelin and possibly also by other types of molecules normally found in their local environment (Brittis and Flanagan, Neuron 2001, 30, pp. 11-14; Jones et al., J. Neurosci. 2002, 22, pp. 2792-2803; Grimpe et al., J. Neurosci. 2002, 22, pp. 3144-3160).

Several myelin inhibitory proteins that are found on oligodendrocytes have been characterized, e.g., NogoA (Chen et al., Nature, 2000, 403, 434-439; Grandpre et al., Nature 2000, 403, 439-444), myelin associated glycoprotein (MAG, McKerracher et al., Neuron 1994, 13, 805-811; Mukhopadhyay et al., Neuron 1994, 13, 757-767) and oligodendrocyte glycoprotein (OM-gp, Mikol and Stefansson, J. Cell. Biol. 1988, 106, 1273-1279). Each of these proteins has been separately shown to be a ligand for the neuronal Nogo receptor-1 (“NgR1”) (Wang et al., Nature 2002, 417, 941-944; Liu et al., Science, 2002, 297, 1190-93; Grandpre et al., Nature 2000, 403, 439-444; Chen et al., Nature, 2000, 403, 434-439; Domeniconi et al., Neuron, 2002, 35, 283-90). Nogo-66 is a 66 amino acid peptide from NogoA having the ability to inhibit neurite outgrowth and cause growth cone collapse. (Fournier et al., Nature 2001, 409, 341-346). Nogo receptor-1 (NgR1) is a leucine rich repeat (LRR) protein that contains eight LRRs flanked by N-terminal and C-terminal cysteine rich domains (LRRNT and LRRCT regions, respectively, and a Ser-, Thr-, Pro-, and Gly-rich stalk region (CT stalk) between the LRRCT and a glycosylphosphatidylinositol (GPI) anchor site. NgR1 forms a signaling complex with LINGO-1 and p75 or Taj (also known as TROY). Upon interaction with an inhibitory protein (e.g., NogoA, MAG and OM-gp), the NgR1 complex transduces signals that lead to growth cone collapse and inhibition of neurite outgrowth. Previous studies have shown that the entire LRR region of Nogo receptor-1, including the C-terminal cap of LRR, LRRCT, is needed for ligand binding, and that the adjacent CT stalk of the Nogo receptor-1 contributes to interaction with its co-receptors.

Axonal damage is a key pathology in many injuries of the central nervous system (CNS), such as spinal cord injury, traumatic brain injury and stroke, as well as in multiple sclerosis (MS). A recently developed strategy for treating CNS injuries and CNS diseases is to interfere with the axonal growth inhibition that occurs through the interaction of myelin proteins with their axonal receptors, such as NgR, LINGO-1, and p75 or Taj. For example, the anti-NogoA antibody IN-1 was shown to improve functional recovery in rats that had undergone spinal cord transection. (Lee et al., Nature Reviews 2003, 2, 1-7.) In addition, a 40 residue peptide known as NEP1-40, an antagonist of NogoA, was shown to attenuate the effects of myelin or Nogo-66 on growth cone collapse and neurite outgrowth, and improved the outcome in vivo following spinal cord injury. (Lee et al., Nature Reviews 2003, 2, 1-7.) Although these reagents have shown great promise in treating injuries to the CNS, there remains a need in the art for additional compounds that inhibit NgR signaling and/or attenuate myelin-mediated growth cone collapse and/or inhibit neurite outgrowth inhibition.

### SUMMARY OF THE INVENTION

The present invention is directed to the use of certain Nogo receptor, polypeptides, including NgR1 and NgR2, and polypeptide fragments thereof for promoting neurite outgrowth, neuronal survival, and axonal regeneration in CNS neurons. The invention features molecules and methods useful for inhibiting neurite outgrowth inhibition, promoting neuronal survival, and/or promoting axonal regeneration in CNS neurons.

In some embodiments, the invention provides an isolated polypeptide fragment of 40 residues or less, comprising amino acids 309 to 344 of SEQ ID NO:2, except for up to three amino acid substitutions.

In some embodiments, the invention provides a polypeptide of the invention that is cyclic. In some embodiments, the cyclic polypeptide further comprises a first molecule linked at the N-terminus and a second molecule linked at the C-terminus; wherein the first molecule and the second molecule are joined to each other to form said cyclic molecule. In some embodiments, the first and second molecules are selected from the group consisting of: a biotin molecule, a cysteine residue, and an acetylated cysteine residue. In some embodiments, the first molecule is a biotin molecule attached to the N-terminus and the second molecule is a cysteine residue attached to the C-terminus of the polypeptide of the invention. In some embodiments, the first molecule is an acetylated cysteine residue attached to the N-terminus and the second molecule is a cysteine residue attached to the C-terminus of the polypeptide of the invention. In some embodiments, the first molecule is an acetylated cysteine residue attached to the N-terminus and the second molecule is a cysteine residue attached to the C-terminus of the polypeptide of the invention. In some embodiments, the C-terminal cysteine has an NH₂ moiety attached.

In some embodiments, the invention provides a polypeptide of the invention wherein at least one cysteine residue is substituted with a different amino acid. In some embodiments, the at least one cysteine residue is C309. In some embodiments, the at least one cysteine residue is C335. In some embodiments, the at least one cysteine residue is at C336. In some embodiments, the at least one cysteine residue is substituted with a different amino acid selected from the group consisting of: alanine, serine, or threonine. In some embodiments, the different amino acid is alanine.

In some embodiments the invention further provides that the polypeptide is fused to a heterologous polypeptide. In some embodiments, the heterologous polypeptide is serum albumin. In some embodiments, the heterologous polypeptide is an Fc region. In some embodiments, the heterologous polypeptide is a signal peptide. In some embodiments, the heterologous polypeptide is a polypeptide tag. In some embodiments, the invention further provides that the Fc region is selected from the group consisting of: an IgA Fc region; an IgD Fc region; an IgG Fc region, an IgEFc region; and an IgM Fc region. In some embodiments, the invention further provides that the polypeptide tag is selected from the group consisting of: FLAG tag; Strep tag; poly-histidine tag; VSV-G tag; influenza virus hemagglutinin (HA) tag; and c-Myc tag.

In some embodiments, the invention provides a polypeptide of the invention attached to one or more polyalkylene glycol moieties. In some embodiments, the invention further provides that the one or more polyalkylene glycol moieties is a polyethylene glycol (PEG) moiety. In some embodiments, the invention further provides a polypeptide of the invention attached to 1 to 5 PEG moieties.

In some embodiments, the invention provides an isolated polynucleotide encoding a polypeptide of the invention. In some embodiments, the invention further provides that the nucleotide sequence is operably linked to an expression control element (e.g. an inducible promoter, a constitutive promoter, or a secretion signal). Additional embodiments include a vector comprising an isolated polynucleotide of the invention and a host cell comprising said vector.

Additional embodiments of the invention include pharmaceutical compositions comprising the polypeptides, polynucleotides, vectors or host cells of the invention and in certain embodiments a pharmaceutically acceptable carrier.

Embodiments of the invention also include methods for promoting neurite outgrowth, comprising contacting a neuron with an agent which includes polypeptides, polynucleotides or compositions of the invention, wherein said agent inhibits Nogo receptor 1-mediated neurite outgrowth inhibition. In certain embodiments, the neuron is in a mammal and in certain embodiments the mammal is a human.

Additional embodiments include a method for inhibiting signal transduction by the NgR1 signaling complex, comprising contacting a neuron with an effective amount of an agent which includes polypeptide, polynucleotides, or compositions of the invention, wherein said agent inhibits signal transduction by the NgR1 signaling complex. In certain embodiments, the neuron is in a mammal and in certain embodiments the mammal is a human.

Other embodiments include a method for treating a central nervous system (CNS) disease, disorder, or injury in a mammal, comprising administering to a mammal in need of treatment an effective amount of an agent which includes polypeptides, polynucleotides, or compositions of the invention, wherein said agent inhibits Nogo Receptor 1-mediated neurite outgrowth inhibition. In certain embodiments, the disease, disorder or injury is selected from the group consisting of multiple sclerosis, ALS, Huntington's disease, Alzheimer's disease, Parkinson's disease, diabetic neuropathy, stroke, traumatic brain injuries, spinal cord injury, optic neuritis, glaucoma, hearing loss, and adrenal leukodystrophy.

### BRIEF DESCRIPTION OF THE DRAWINGS

FIG. 1 shows the human FL-NgR1 sequence excluding the GPI domain (SEQ ID NO:22). The LRRNT region is represented by amino acids 27-73. The 8 LRR regions are represented by amino acids 74-249. The LRRCT domain is represented by amino acids 250-310. The extended LRRCT region is represented by amino acids 311-337. The stalk region is represented by amino acids 338-438. Disulfide bonds determined in this study are indicated with a black line joining particular Cys residues. Cys residues in the free thiol form are highlighted in gray. A hydroxyproline (Hyp) residue is double underlined; glycosylation sites are underlined. Signal peptide and flag sequences are not shown. A schematic diagram of the human FL-NgR1 is shown below the sequence.

FIG. 2A shows a SDS PAGE of various NgRI proteins.

FIG. 2B shows a size exclusion chromatography (SEC) profile of FL-NgR1.

FIG. 2C shows an ELISA plot, using an anti-NgR1 antibody to block the binding of AP-OMgp and AP-Lingo-1 to FL-NgR1.

FIG. 3 shows tryptic peptide maps of pyridylethylated FL-NgR1. Upper panel, non-reduced digest; lower panel, reduced digest.

FIG. 4 shows a MS/MS spectrum of the partially reduced peptide T1 containing a NES group (SEQ ID NO:18).

FIG. 5 shows a deconvoluted mass spectrum of Peak 2 from the endo-Asp-N treated disulfide-linked tryptic peptides cluster T21/T24/T28/T30 from FL-NgR1. The y and b ions are due to in-source fragmentation. The figure shows a partial sequence of peptide T21 (SEQ ID NO:19) and the full sequence of peptide T24 (SEQ ID NO:20).

FIG. 6 shows a total Ion Chromatogram (TIC) of partially reduced, NEM-alkylated disulfide-linked peptides cluster T21/T24/T28/T30 from FL-NgR1. Identities of components in each peak are listed in Table 3.

FIG. 7 shows a MS/MS spectrum of the peptide T30 containing residues 335-343 with a NES group (SEQ ID NO:21) which was generated from reduction of partially reduced disulfide-linked tryptic peptide 335-343 and tryptic peptide 301-323.

FIG. 8 shows possible disulfide linkages in peptide T21/T24/T28/T30 cluster. The figure shows the full sequence of peptides T24 (SEQ ID NO:20), T21 (SEQ ID NO:27), T30 (SEQ ID NO:28), T28 (SEQ ID NO:29).

FIG. 9 shows the disulfide linkages in peptide cluster T21/T24/T28/T30. The figure shows the full sequence of peptides T24 (SEQ ID NO:20), T21 (SEQ ID NO:27), T30 (SEQ ID NO:28), T28 (SEQ ID NO:29).

FIG. 10 shows the protein sequence alignment of different NgR forms.

FIG. 11 shows the tryptic peptide maps of pyridylethylated rat NgR1(310). Only Cys-containing peptides that form a disulfide bond are labeled on the maps. The figure shows peptides T21 (SEQ ID NO:30), T18 (SEQ ID NO:31) and T25 (SEQ ID NO:32) of rat NgR1.

FIG. 12 shows disulfide structures in NgR2 and NgR1 made from different constructs. The figures shows amino acids 27-473 of SEQ ID NO:2 (human NgR1), amino acids 27-473 of SEQ ID NO:23 (rat NgR1) and amino acids 31-420 of SEQ ID NO:24 (human NgR2).

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

In order to further define this invention, the following terms and definitions are provided.

It is to be noted that the term “a” or “an” entity, refers to one or more of that entity; for example, “an immunoglobulin molecule,” is understood to represent one or more immunoglobulin molecules. As such, the terms “a” (or “an”), “one or more,” and “at least one” can be used interchangeably herein.

Throughout this specification and claims, the word “comprise,” or variations such as “comprises” or “comprising,” indicate the inclusion of any recited integer or group of integers but not the exclusion of any other integer or group of integers.

As used herein, the term “consists of,” or variations such as “consist of” or “consisting of,” as used throughout the specification and claims, indicate the inclusion of any recited integer or group of integers, but that no additional integer or group of integers may be added to the specified method, structure or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

In the present invention, a “polypeptide fragment” refers to a short amino acid sequence of a larger polypeptide. Protein fragments may be “free-standing,” or comprised within a larger polypeptide of which the fragment forms a part of region. Representative examples of polypeptide fragments of the invention, include, for example, fragments comprising about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, and about 100 amino acids or more in length.

The terms “fragment,” “variant,” “derivative” and “analog” when referring to a polypeptide of the present invention include any polypeptide which retains at least some biological activity. Polypeptides as described herein may include fragment, variant, or derivative molecules therein without limitation, so long as the polypeptide still serves its function. NgR1 polypeptides and polypeptide fragments of the present invention may include proteolytic fragments, deletion fragments and in particular, fragments which more easily reach the site of action when delivered to an animal. Polypeptide fragments further include any portion of the polypeptide which comprises an antigenic or immunogenic epitope of the native polypeptide, including linear as well as three-dimensional epitopes. NgR1 polypeptides and polypeptide fragments of the present invention may comprise variant regions, including fragments as described above, and also polypeptides with altered amino acid sequences due to amino acid substitutions, deletions, or insertions. Variants may occur naturally, such as an allelic variant. By an “allelic variant” is intended alternate forms of a gene occupying a given locus on a chromosome of an organism. Genes II, Lewin, B., ed., John Wiley & Sons, New York (1985). Non-naturally occurring variants may be produced using art-known mutagenesis techniques. NgR1 polypeptides and polypeptide fragments of the invention may comprise conservative or non-conservative amino acid substitutions, deletions or additions. NgR1 polypeptides and polypeptide fragments of the present invention may also include derivative molecules. Variant polypeptides may also be referred to herein as “polypeptide analogs.” As used herein a “derivative” of a polypeptide or a polypeptide fragment refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

As used herein the term “disulfide bond” includes the covalent bond formed between two sulfur atoms. The amino acid cysteine comprises a thiol group that can form a disulfide bond or bridge with a second thiol group.

As used herein, “fusion protein” means a protein comprising a first polypeptide linearly connected, via peptide bonds, to a second, polypeptide. The first polypeptide and the second polypeptide may be identical or different, and they may be directly connected, or connected via a peptide linker (see below).

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can contain the nucleotide sequence of the full length cDNA sequence, including the untranslated 5′ and 3′ sequences, the coding sequences. A polynucleotide may comprise a conventional phosphodiester bond or a non-conventional bond (e.g., an amide bond, such as found in peptide nucleic acids (PNA)). The polynucleotide can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. As used herein, In addition, the polynucleotides can be composed of triple-stranded regions comprising RNA or DNA or both RNA and DNA. polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The term “nucleic acid” refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an NgR polypeptide or polypeptide fragment of the invention contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding an NgR polypeptide or polypeptide fragment of the present invention. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

A “linker” sequence is a series of one or more amino acids separating two polypeptide coding regions in a fusion protein. A typical linker comprises at least 5 amino acids. Additional linkers comprise at least 10 or at least 15 amino acids. In certain embodiments, the amino acids of a peptide linker are selected so that the linker is hydrophilic. The linker (Gly-Gly-Gly-Gly-Ser)₃ (SEQ ID NO:3) is a preferred linker that is widely applicable to many antibodies as it provides sufficient flexibility. Other linkers include Glu Ser Gly Arg Ser Gly Gly Gly Gly Ser Gly Gly Gly Gly Ser (SEQ ID NO:4), Glu Gly Lys Ser Ser Gly Ser Gly Ser Glu Ser Lys Ser Thr (SEQ ID NO:5), Glu Gly Lys Ser Ser Gly Ser Gly Ser Glu Ser Lys Ser Thr Gln (SEQ ID NO:6), Glu Gly Lys Ser Ser Gly Ser Gly Ser Glu Ser Lys Val Asp (SEQ ID NO:7), Gly Ser Thr Ser Gly Ser Gly Lys Ser Ser Glu Gly Lys Gly (SEQ ID NO:8), Lys Glu Ser Gly Ser Val Ser Ser Glu Gln Leu Ala Gln Phe Arg Ser Leu Asp (SEQ ID NO:9), and Glu Ser Gly Ser Val Ser Ser Glu Glu Leu Ala Phe Arg Ser Leu Asp (SEQ ID NO:10). Examples of shorter linkers include fragments of the above linkers, and examples of longer linkers include combinations of the linkers above, combinations of fragments of the linkers above, and combinations of the linkers above with fragments of the linkers above.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product, and the translation of such mRNA into polypeptide(s), as well as any processes which regulate either transcription or translation. If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

As used herein, phrases such as “a subject that would benefit from administration of an NgR polypeptide or polypeptide fragment of the present invention” and “an animal in need of treatment” includes subjects, such as mammalian subjects, that would benefit from administration of an NgR polypeptide or polypeptide fragment of the present invention used, e.g., for detection (e.g., for a diagnostic procedure) and/or for treatment, i.e., palliation or prevention of a disease such as MS, with an NgR polypeptide or polypeptide fragment of the present invention. As described in more detail herein, the polypeptide or polypeptide fragment can be used in unconjugated form or can be conjugated, e.g., to a drug, prodrug, or an isotope.

As used herein, a “therapeutically effective amount” refers to an amount effective, at dosages and for periods of time necessary, to achieve the desired therapeutic result. A therapeutic result may be, e.g., lessening of symptoms, prolonged survival, improved mobility, and the like. A therapeutic result need not be a “cure”.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The invention is directed to certain NgR1 polypeptides and polypeptide fragments that promote neuronal survival, neurite outgrowth and axonal regeneration of neurons, for example, CNS neurons. For example, the present invention provides NgR1 polypeptides and polypeptide fragments which stimulate axonal growth under conditions in which axonal growth is normally inhibited. Thus, the NgR1 polypeptides and polypeptide fragments of the invention are useful in treating injuries, diseases or disorders that can be alleviated by promoting neuronal survival, or by the stimulation of axonal growth or regeneration in the CNS.

Exemplary CNS diseases, disorders or injuries include, but are not limited to, multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Globoid cell Leucodystrophy (Krabbe's disease) and Wallerian Degeneration, optic neuritis, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, and Bell's palsy. Among these diseases, MS is the most widespread, affecting approximately 2.5 million people worldwide.

### NgR Polypeptides and Polypeptide Fragments

The present invention is directed to certain Nogo receptor polypeptides, including NgR1 and NgR2, and polypeptide fragments useful, e.g., for promoting neurite outgrowth, promoting neuronal survival, promoting axonal survival, or inhibiting signal transduction by the NgR signaling complex. Typically, the polypeptides and polypeptide fragments of the invention act to block NgR-mediated inhibition of neuronal survival, neurite outgrowth or axonal regeneration of central nervous system (CNS) neurons.

The human NgR1 polypeptide is shown below as SEQ ID NO:2 and is accession number NP_{—}075380 in Genbank.

Full-Length Human NgR1 (SEQ ID NO:2):
MKRASAGGSRLLAWVLWLQAWQVAAPCPGACVCYNEPKVTTSCPQQGLQA
VPVGIPAASQRIFLHGNRISHVPAASFRACRNLTILWLHSNVLARIDAAA
FTGLALLEQLDLSDNAQLRSVDPATFHGLGRLHTLHLDRCGLQELGPGLF
RGLAALQYLYLQDNALQALPDDTFRDLGNLTHLFLHGNRISSVPERAFRG
LHSLDRLLLHQNRVAHVHPHAFRDLGRLMTLYLFANNLSALPTEALAPLR
ALQYLRLNDNPWVCDCRARPLWAWLQKFRGSSSEVPCSLPQRLAGRDLKR
LAANDLQGCAVATGPYHPIWTGRATDEEPLGLPKCCQPDAADKASVLEPG
RPASAGNALKGRVPPGDSPPGNGSGPRHINDSPFGTLPGSAEPPLTAVRP
EGSEPPGFPTSGPRRRPGCSRKNRTRSHCRLGQAGSGGGGTGDSEGSGAL
PSLTCSLTPLGLALVLWTVLGPC.

The rat NgR1 polypeptide is shown below as SEQ ID NO:23 and is accession number

Full-Length Rat NgR1 (SEQ ID NO:23):
MKRASSGGSRLLAWVLWLQAWRVATPCPGACVCYNEPKVTTSCPQQGLQA
VPTGIPAASQRIFLHGNRISHVPAASFQSCRNLTILWLHSNALARIDAAA
FTGLTLLEQLDLSDNAQLHVVDPTTFHGLGHLHTLHLDRCGLRELGPGLF
RGLAALQYLYLQDNNLQALPDNTFRDLGNLTHLFLHGNRIPSVPEHAFRG
LHSLDRLLLHQNHVARVHPHAFRDLGRLMTLYLFANNLSMLPAEVLMPLR
SLQYLRLNDNPWVCDCRARPLWAWLQKFRGSSSEVPCNLPQRLADRDLKR
LAASDLEGCAVASGPFRPIQTSQLTDEELLSLPKCCQPDAADKASVLEPG
RPASAGNALKGRVPPGDTPPGNGSGPRHINDSPFGTLPSSAEPPLTALRP
GGSEPPGLPTTGPRRRPGCSRKNRTRSHCRLGQAGSGASGTGDAEGSGAL
PALACSLAPLGLALVLWTVLGPC.

The human NgR2 polypeptide is shown below as SEQ ID NO:24 and is accession number NP_{—}848665 in Genbank.

Full-Length Human NgR2 (SEQ ID NO:24):
MLPGLRRLLQAPASACLLLMLLALPLAAPSCPMLCTCYSSPPTVSCQANN
FSSVPLSLPPSTQRLFLQNNLIRTLRPGTFGSNLLTLWLFSNNLSTIYPG
TFRHLQALEELDLGDNRHLRSLEPDTFQGLERLQSLHLYRCQLSSLPGNI
FRGLVSLQYLYLQENSLLHLQDDLFADLANLSHLFLHGNRLRLLTEHVFR
GLGSLDRLLLHGNRLQGVHRAAFRGLSRLTILYLFNNSLASLPGEALADL
PSLEFLRLNANPWACDCRARPLWAWFQRARVSSSDVTCATPPERQGRDLR
ALREADFQACPPAAPTRPGSRARGNSSSNHLYGVAEAGAPPADPSTLYRD
LPAEDSRGRQGGDAPTEDDYWGGYGGEDQRGEQMCPGAACQAPPDSRGPA
LSAGLPSPLLCLLLLVPHHL

In one embodiment, the present invention provides an isolated polypeptide fragment of 40 residues or less, where the polypeptide fragment comprises an amino acid sequence identical to amino acids 309 to 344 of SEQ ID NO:2, except for up to one, two, three, four, ten, or twenty individual amino acid substitutions.

By “an NgR1 reference amino acid sequence,” “an NgR2 reference amino acid sequence,” or “reference amino acid sequence” is meant the specified sequence without the introduction of any amino acid substitutions. As one of ordinary skill in the art would understand, if there are no substitutions, the “isolated polypeptide” of the invention comprises an amino acid sequence which is identical to the reference amino acid sequence.

In one embodiment, the present invention provides an isolated polypeptide fragment of 40 residues or less, where the polypeptide fragment comprises an amino acid sequence identical to amino acids 309 to 344 of SEQ ID NO:2, except for up to three individual amino acid substitutions.

In another embodiment, the present invention provides an isolated polypeptide fragment of 40 residues or less, where the polypeptide fragment comprises, consists of or consists essentially of an amino acid sequence identical to amino acids 309 to 344 of SEQ ID NO:2, except for one, two or three amino acid substitutions.

Exemplary amino acid substitutions for polypeptide fragments according to this embodiment include substitutions of individual cysteine residues in the polypeptides of the invention with different amino acids. The cysteine residues in the polypeptides of the invention may be substituted with any heterologous amino acid. Which different amino acid is used depends on a number of criteria, for example, the effect of the substitution on the conformation of the polypeptide fragment, the charge of the polypeptide fragment, or the hydrophilicity of the polypeptide fragment. Amino acid substitutions for the amino acids of the polypeptides of the invention and the reference amino acid sequence can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Typical amino acids to substitute for cysteines in the reference amino acid include alanine, serine, threonine, in particular, alanine. Making such substitutions through engineering of a polynucleotide encoding the polypeptide fragment is well within the routine expertise of one of ordinary skill in the art. In certain embodiments, the cysteine is substituted with a small uncharged amino acid which is least likely to alter the three dimensional conformation of the polypeptide, e.g., alanine, serine, threonine. In certain embodiments, the substituted amino acid is alanine.

In another embodiment, the present invention provides an isolated polypeptide of the invention wherein at least one cysteine residue is substituted with a different amino acid. Cysteine residues that can substituted include but are not limited to C27, C31, C33, C43, C80, C140, C264, C266, C287, C309, C335, C336, C419, C429, C455 and C473. The present invention further provides an isolated polypeptide fragment of 40 residues or less, where the polypeptide fragment comprises an amino acid sequence identical to amino acids 309 to 344 of SEQ ID NO:2, except that: C309 is substituted, C335 is substituted, C336 is substituted, C309 and C335 are substituted, C309 and C336 are substituted, C335 and C336 are substituted, or C309, C335, and C336 are substituted.

In one aspect, the invention includes a polypeptide comprising two or more polypeptide fragments as described above in a fusion protein, as well as fusion proteins comprising a polypeptide fragment as described above fused to a heterologous amino acid sequence. The invention further encompasses variants, analogs, or derivatives of polypeptide fragments as described above.

In the present invention, a polypeptide can be composed of amino acids joined to each other by peptide bonds or modified peptide bonds, i.e., peptide isosteres, and may contain amino acids other than the 20 gene-encoded amino acids (e.g. non-naturally occurring amino acids). The polypeptides of the present invention may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W. H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).).

Polypeptides described herein may be cyclic. Cyclization of the polypeptides reduces the conformational freedom of linear peptides and results in a more structurally constrained molecule. Many methods of peptide cyclization are known in the art. For example, “backbone to backbone” cyclization by the formation of an amide bond between the N-terminal and the C-terminal amino acid residues of the peptide. The “backbone to backbone” cyclization method includes the formation of disulfide bridges between two ω-thio amino acid residues (e.g. cysteine, homocysteine). Certain peptides of the present invention include modifications on the N- and C-terminus of the peptide to form a cyclic polypeptide. Such modifications include, but are not limited, to cysteine residues, acetylated cysteine residues, cysteine residues with a NH₂ moiety and biotin. Other methods of peptide cyclization are described in Li & Roller. Curr. Top. Med. Chem. 3:325-341 (2002), which is incorporated by reference herein in its entirety.

In methods of the present invention, an NgR1 polypeptide or polypeptide fragment of the invention can be administered directly as a preformed polypeptide, or indirectly through a nucleic acid vector. In some embodiments of the invention, an NgR1 polypeptide or polypeptide fragment of the invention is administered in a treatment method that includes: (1) transforming or transfecting an implantable host cell with a nucleic acid, e.g., a vector, that expresses an NgR1 polypeptide or polypeptide fragment of the invention; and (2) implanting the transformed host cell into a mammal, at the site of a disease, disorder or injury. For example, the transformed host cell can be implanted at the site of a chronic lesion of MS. In some embodiments of the invention, the implantable host cell is removed from a mammal, temporarily cultured, transformed or transfected with an isolated nucleic acid encoding an NgR1 polypeptide or polypeptide fragment of the invention, and implanted back into the same mammal from which it was removed. The cell can be, but is not required to be, removed from the same site at which it is implanted. Such embodiments, sometimes known as ex vivo gene therapy, can provide a continuous supply of the NgR1 polypeptide or polypeptide fragment of the invention, localized at the site of action, for a limited period of time.

Additional exemplary NgR polypeptides of the invention and methods and materials for obtaining these molecules for practicing the present invention are described below.

### Fusion Proteins and Conjugated Polypeptides

Some embodiments of the invention involve the use of an NgR polypeptide that is not the full-length NgR protein, e.g., polypeptide fragments of NgR, fused to a heterologous polypeptide moiety to form a fusion protein. Such fusion proteins can be used to accomplish various objectives, e.g., increased serum half-life, improved bioavailability, in vivo targeting to a specific organ or tissue type, improved recombinant expression efficiency, improved host cell secretion, ease of purification, and higher avidity. Depending on the objective(s) to be achieved, the heterologous moiety can be inert or biologically active. Also, it can be chosen to be stably fused to the NgR polypeptide moiety of the invention or to be cleavable, in vitro or in vivo. Heterologous moieties to accomplish these other objectives are known in the art.

As an alternative to expression of a fusion protein, a chosen heterologous moiety can be preformed and chemically conjugated to the NgR polypeptide moiety of the invention. In most cases, a chosen heterologous moiety will function similarly, whether fused or conjugated to the NgR polypeptide moiety. Therefore, in the following discussion of heterologous amino acid sequences, unless otherwise noted, it is to be understood that the heterologous sequence can be joined to the NgR polypeptide moiety in the form of a fusion protein or as a chemical conjugate.

Pharmacologically active polypeptides such as NgR polypeptides may exhibit rapid in vivo clearance, necessitating large doses to achieve therapeutically effective concentrations in the body. In addition, polypeptides smaller than about 60 kDa potentially undergo glomerular filtration, which sometimes leads to nephrotoxicity. Fusion or conjugation of relatively small polypeptides such as polypeptide fragments of the NgR signaling domain can be employed to reduce or avoid the risk of such nephrotoxicity. Various heterologous amino acid sequences, i.e., polypeptide moieties or “carriers,” for increasing the in vivo stability, i.e., serum half-life, of therapeutic polypeptides are known. Examples include serum albumins such as, e.g., bovine serum albumin (BSA) or human serum albumin (HSA).

Due to its long half-life, wide in vivo distribution, and lack of enzymatic or immunological function, essentially full-length human serum albumin (HSA), or an HSA fragment, is commonly used as a heterologous moiety. Through application of methods and materials such as those taught in Yeh et al., Proc. Natl. Acad. Sci. USA, 89:1904-08 (1992) and Syed et al, Blood 89:3243-52 (1997), HSA can be used to form a fusion protein or polypeptide conjugate that displays pharmacological activity by virtue of the NgR polypeptide moiety while displaying significantly increased in vivo stability, e.g., 10-fold to 100-fold higher. The C-terminus of the HSA can be fused to the N-terminus of the NgR polypeptide moiety. Since HSA is a naturally secreted protein, the HSA signal sequence can be exploited to obtain secretion of the fusion protein into the cell culture medium when the fusion protein is produced in a eukaryotic, e.g., mammalian, expression system.

In certain embodiments, NgR polypeptides for use in the methods of the present invention further comprise a targeting moiety. Targeting moieties include a protein or a peptide which directs localization to a certain part of the body, for example, to the brain or compartments therein. In certain embodiments, NgR polypeptides for use in the methods of the present invention are attached or fused to a brain targeting moiety. The brain targeting moieties are attached covalently (e.g., direct, translational fusion, or by chemical linkage either directly or through a spacer molecule, which can be optionally cleavable) or non-covalently attached (e.g., through reversible interactions such as avidin:biotin, protein A:IgG, etc.). In other embodiments, the NgR polypeptides for use in the methods of the present invention thereof are attached to one more brain targeting moieties. In additional embodiments, the brain targeting moiety is attached to a plurality of NgR polypeptides for use in the methods of the present invention.

A brain targeting moiety associated with a NgR polypeptide enhances brain delivery of such a NgR polypeptide. A number of polypeptides have been described which, when fused to a protein or therapeutic agent, delivers the protein or therapeutic agent through the blood-brain barrier (BBB). Non-limiting examples include the single domain antibody FC5 (Abulrob et al. (2005) J. Neurochem. 95, 1201-1214); mAB 83-14, a monoclonal antibody to the human insulin receptor (Pardridge et al. (1995) Pharmacol. Res. 12, 807-816); the B2, B6 and B8 peptides binding to the human transferrin receptor (hTfR) (Xia et al. (2000) J. Virol. 74, 11359-11366); the OX26 monoclonal antibody to the transferrin receptor (Pardridge et al. (1991) J. Pharmacol Exp. Ther. 259, 66-70); diptheria toxin conjugates (see, for e.g., Gaillard et al., International Congress Series 1277:185-198 (2005); and SEQ ID NOs: 1-18 of U.S. Pat. No. 6,306,365. The contents of the above references are incorporated herein by reference in their entirety.

Enhanced brain delivery of a NgR composition is determined by a number of means well established in the art. For example, administering to an animal a radioactively labelled NgR polypeptide linked to a brain targeting moiety; determining brain localization; and comparing localization with an equivalent radioactively labelled NgR polypeptide that is not associated with a brain targeting moiety. Other means of determining enhanced targeting are described in the above references.

Some embodiments of the invention employ an NgR polypeptide moiety fused to a hinge and Fc region, i.e., the C-terminal portion of an Ig heavy chain constant region. In some embodiments, amino acids in the hinge region may be substituted with different amino acids. Exemplary amino acid substitutions for the hinge region according to these embodiments include substitutions of individual cysteine residues in the hinge region with different amino acids. Any different amino acid may be substituted for a cysteine in the hinge region. Amino acid substitutions for the amino acids of the polypeptides of the invention and the reference amino acid sequence can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Typical amino acids to substitute for cysteines in the reference amino acid include alanine, serine, threonine, in particular, serine and alanine. Making such substitutions through engineering of a polynucleotide encoding the polypeptide fragment is well within the routine expertise of one of ordinary skill in the art.

Potential advantages of an NgR-polypeptide-Fc fusion include solubility, in vivo stability, and multivalency, e.g., dimerization. The Fc region used can be an IgA, IgD, or IgG Fc region (hinge-CH2-CH3). Alternatively, it can be an IgE or IgM Fc region (hinge-CH2-CH3-CH4). An IgG Fc region is generally used, e.g., an IgG1 Fc region or IgG4 Fc region. Materials and methods for constructing and expressing DNA encoding Fc fusions are known in the art and can be applied to obtain fusions without undue experimentation. Some embodiments of the invention employ a fusion protein such as those described in Capon et al, U.S. Pat. Nos. 5,428,130 and 5,565,335.

The signal sequence is a polynucleotide that encodes an amino acid sequence that initiates transport of a protein across the membrane of the endoplasmic reticulum. Signal sequences useful for constructing an immunofusin include antibody light chain signal sequences, e.g., antibody 14.18 (Gillies et al., J. Immunol. Meth., 125:191-202 (1989)), antibody heavy chain signal sequences, e.g., the MOPC141 antibody heavy chain signal sequence (Sakano et al., Nature 286:5774 (1980)). Alternatively, other signal sequences can be used. See, e.g., Watson, Nucl. Acids Res. 12:5145 (1984). The signal peptide is usually cleaved in the lumen of the endoplasmic reticulum by signal peptidases. This results in the secretion of a immunofusin protein containing the Fc region and the NgR polypeptide moiety.

In some embodiments, the DNA sequence may encode a proteolytic cleavage site between the secretion cassette and the NgR polypeptide moiety. Such a cleavage site may provide, e.g., for the proteolytic cleavage of the encoded fusion protein, thus separating the Fc domain from the target protein. Useful proteolytic cleavage sites include amino acid sequences recognized by proteolytic enzymes such as trypsin, plasmin, thrombin, factor Xa, or enterokinase K.

The secretion cassette can be incorporated into a replicable expression vector. Useful vectors include linear nucleic acids, plasmids, phagemids, cosmids and the like. An exemplary expression vector is pdC, in which the transcription of the immunofusin DNA is placed under the control of the enhancer and promoter of the human cytomegalovirus. See, e.g., Lo et al., Biochim. Biophys. Acta 1088:712 (1991); and Lo et al., Protein Engineering 11:495-500 (1998). An appropriate host cell can be transformed or transfected with a DNA that encodes an NgR1 polypeptide or polypeptide fragment of the invention and used for the expression and secretion of the polypeptide. Host cells that are typically used include immortal hybridoma cells, myeloma cells, 293 cells, Chinese hamster ovary (CHO) cells, Hela cells, and COS cells.

Fully intact, wild-type Fc regions display effector functions that normally are unnecessary and undesired in an Fc fusion protein used in the methods of the present invention. Therefore, certain binding sites typically are deleted from the Fc region during the construction of the secretion cassette. For example, since coexpression with the light chain is unnecessary, the binding site for the heavy chain binding protein, Bip (Hendershot et al., Immunol. Today 8:111-14 (1987)), is deleted from the CH2 domain of the Fc region of IgE, such that this site does not interfere with the efficient secretion of the immunofusin. Transmembrane domain sequences, such as those present in IgM, also are generally deleted.

The IgG1 Fc region is most often used. Alternatively, the Fc region of the other subclasses of immunoglobulin gamma (gamma-2, gamma-3 and gamma-4) can be used in the secretion cassette. The IgG1 Fc region of immunoglobulin gamma-1 is generally used in the secretion cassette and includes at least part of the hinge region, the CH2 region, and the CH3 region. In some embodiments, the Fc region of immunoglobulin gamma-1 is a CH2-deleted-Fc, which includes part of the hinge region and the CH3 region, but not the CH2 region. A CH2-deleted-Fc has been described by Gillies et al., Hum. Antibod. Hybridomas 1:47 (1990). In some embodiments, the Fc region of one of IgA, IgD, IgE, or IgM, is used.

NgR-polypeptide-moiety-Fc fusion proteins can be constructed in several different configurations. In one configuration the C-terminus of the NgR polypeptide moiety is fused directly to the N-terminus of the Fc hinge moiety. In a slightly different configuration, a short polypeptide, e.g., 2-10 amino acids, is incorporated into the fusion between the N-terminus of the NgR polypeptide moiety and the C-terminus of the Fc moiety. In the alternative configuration, the short polypeptide is incorporated into the fusion between the C-terminus of the NgR polypeptide moiety and the N-terminus of the Fc moiety. Such a linker provides conformational flexibility, which may improve biological activity in some circumstances. If a sufficient portion of the hinge region is retained in the Fc moiety, the NgR-polypeptide-moiety-Fc fusion will dimerize, thus forming a divalent molecule. A homogeneous population of monomeric Fc fusions will yield monospecific, bivalent dimers. A mixture of two monomeric Fc fusions each having a different specificity will yield bispecific, bivalent dimers.

Any of a number of cross-linkers that contain a corresponding amino-reactive group and thiol-reactive group can be used to link an NgR1 polypeptide or polypeptide fragment of the invention to serum albumin. Examples of suitable linkers include amine reactive cross-linkers that insert a thiol-reactive maleimide, e.g., SMCC, AMAS, BMPS, MBS, EMCS, SMPB, SMPH, KMUS, and GMBS. Other suitable linkers insert a thiol-reactive haloacetate group, e.g., SBAP, SIA, SIAB. Linkers that provide a protected or non-protected thiol for reaction with sulfhydryl groups to product a reducible linkage include SPDP, SMPT, SATA, and SATP. Such reagents are commercially available (e.g., Pierce Chemical Company, Rockford, Ill.).

Conjugation does not have to involve the N-terminus of an NgR1 polypeptide or polypeptide fragment of the invention or the thiol moiety on serum albumin. For example, NgR-polypeptide-albumin fusions can be obtained using genetic engineering techniques, wherein the NgR polypeptide moiety is fused to the serum albumin gene at its N-terminus, C-terminus, or both.

NgR polypeptides of the invention can be fused to a polypeptide tag. The term “polypeptide tag,” as used herein, is intended to mean any sequence of amino acids that can be attached to, connected to, or linked to an NgR polypeptide and that can be used to identify, purify, concentrate or isolate the NgR polypeptide. The attachment of the polypeptide tag to the NgR polypeptide may occur, e.g., by constructing a nucleic acid molecule that comprises: (a) a nucleic acid sequence that encodes the polypeptide tag, and (b) a nucleic acid sequence that encodes an NgR polypeptide. Exemplary polypeptide tags include, e.g., amino acid sequences that are capable of being post-translationally modified, e.g., amino acid sequences that are biotinylated. Other Exemplary polypeptide tags include, e.g., amino acid sequences that are capable of being recognized and/or bound by an antibody (or fragment thereof) or other specific binding reagent. Polypeptide tags that are capable of being recognized by an antibody (or fragment thereof) or other specific binding reagent include, e.g., those that are known in the art as “epitope tags.” An epitope tag may be a natural or an artificial epitope tag. Natural and artificial epitope tags are known in the art, including, e.g., artificial epitopes such as FLAG, Strep, or poly-histidine peptides. FLAG peptides include the sequence Asp-Tyr-Lys-Asp-Asp-Asp-Asp-Lys (SEQ ID NO:11) or Asp-Tyr-Lys-Asp-Glu-Asp-Asp-Lys (SEQ ID NO:12) (Einhauer, A. and Jungbauer, A., J. Biochem. Biophys. Methods 49:1-3: 455-465 (2001)). The Strep epitope has the sequence Ala-Trp-Arg-His-Pro-Gln-Phe-Gly-Gly (SEQ ID NO:13). The VSV-G epitope can also be used and has the sequence Tyr-Thr-Asp-Ile-Glu-Met-Asn-Arg-Leu-Gly-Lys (SEQ ID NO:14). Another artificial epitope is a poly-His sequence having six histidine residues (His-His-His-His-His-His (SEQ ID NO:15). Naturally-occurring epitopes include the influenza virus hemagglutinin (HA) sequence Tyr-Pro-Tyr-Asp-Val-Pro-Asp-Tyr-Ala-Ile-Glu-Gly-Arg (SEQ ID NO:16) recognized by the monoclonal antibody 12CA5 (Murray et al., Anal. Biochem. 229:170-179 (1995)) and the eleven amino acid sequence from human c-myc (Myc) recognized by the monoclonal antibody 9E10 (Glu-Gln-Lys-Leu-Leu-Ser-Glu-Glu-Asp-Leu-Asn (SEQ ID NO:17) (Manstein et al., Gene 162:129-134 (1995)). Another useful epitope is the tripeptide Glu-Glu-Phe which is recognized by the monoclonal antibody YL ½. (Stammers et al. FEBS Lett. 283:298-302 (1991)).

In certain embodiments, the NgR polypeptide and the polypeptide tag may be connected via a linking amino acid sequence. As used herein, a “linking amino acid sequence” may be an amino acid sequence that is capable of being recognized and/or cleaved by one or more proteases. Amino acid sequences that can be recognized and/or cleaved by one or more proteases are known in the art. Exemplary amino acid sequences are those that are recognized by the following proteases: factor VIIa, factor Ixa, factor Xa, APC, t-PA, u-PA, trypsin, chymotrypsin, enterokinase, pepsin, cathepsin B,H,L,S,D, cathepsin G, renin, angiotensin converting enzyme, matrix metalloproteases (collagenases, stromelysins, gelatinases), macrophage elastase, Cir, and Cis. The amino acid sequences that are recognized by the aforementioned proteases are known in the art. Exemplary sequences recognized by certain proteases can be found, e.g., in U.S. Pat. No. 5,811,252.

Polypeptide tags can facilitate purification using commercially available chromatography media.

In some embodiments of the invention, an NgR polypeptide fusion construct is used to enhance the production of an NgR polypeptide moiety in bacteria. In such constructs a bacterial protein normally expressed and/or secreted at a high level is employed as the N-terminal fusion partner of an NgR1 polypeptide or polypeptide fragment of the invention. See, e.g., Smith et al., Gene 67:31 (1988); Hopp et al., Biotechnology 6:1204 (1988); La Vallie et al., Biotechnology 11:187 (1993).

By fusing an NgR polypeptide moiety at the amino and carboxy termini of a suitable fusion partner, bivalent or tetravalent forms of an NgR1 polypeptide or polypeptide fragment of the invention can be obtained. For example, an NgR polypeptide moiety can be fused to the amino and carboxy termini of an Ig moiety to produce a bivalent monomeric polypeptide containing two NgR polypeptide moieties. Upon dimerization of two of these monomers, by virtue of the Ig moiety, a tetravalent form of an NgR polypeptide is obtained. Such multivalent forms can be used to achieve increased binding affinity for the target. Multivalent forms of an NgR1 polypeptide or polypeptide fragment of the invention also can be obtained by placing NgR polypeptide moieties in tandem to form concatamers, which can be employed alone or fused to a fusion partner such as Ig or HSA.

### Conjugated Polymers (Other Than Polypeptides)

Some embodiments of the invention involve an NgR1 polypeptide or polypeptide fragment of the invention wherein one or more polymers are conjugated (covalently linked) to the NgR polypeptide. Examples of polymers suitable for such conjugation include polypeptides (discussed above), sugar polymers and polyalkylene glycol chains. Typically, but not necessarily, a polymer is conjugated to the NgR1 polypeptide or polypeptide fragment of the invention for the purpose of improving one or more of the following: solubility, stability, or bioavailability.

The class of polymer generally used for conjugation to an NgR1 polypeptide or polypeptide fragment of the invention is a polyalkylene glycol. Polyethylene glycol (PEG) is most frequently used. PEG moieties, e.g., 1, 2, 3, 4 or 5 PEG polymers, can be conjugated to each NgR polypeptide to increase serum half life, as compared to the NgR polypeptide alone. PEG moieties are non-antigenic and essentially biologically inert. PEG moieties used in the practice of the invention may be branched or unbranched.

The number of PEG moieties attached to the NgR polypeptide and the molecular weight of the individual PEG chains can vary. In general, the higher the molecular weight of the polymer, the fewer polymer chains attached to the polypeptide. Usually, the total polymer mass attached to an NgR polypeptide or polypeptide fragment is from 20 kDa to 40 kDa. Thus, if one polymer chain is attached, the molecular weight of the chain is generally 20-40 kDa. If two chains are attached, the molecular weight of each chain is generally 10-20 kDa. If three chains are attached, the molecular weight is generally 7-14 kDa.

The polymer, e.g., PEG, can be linked to the NgR polypeptide through any suitable, exposed reactive group on the polypeptide. The exposed reactive group(s) can be, e.g., an N-terminal amino group or the epsilon amino group of an internal lysine residue, or both. An activated polymer can react and covalently link at any free amino group on the NgR polypeptide. Free carboxylic groups, suitably activated carbonyl groups, hydroxyl, guanidyl, imidazole, oxidized carbohydrate moieties and mercapto groups of the NgR polypeptide (if available) also can be used as reactive groups for polymer attachment.

In a conjugation reaction, from about 1.0 to about 10 moles of activated polymer per mole of polypeptide, depending on polypeptide concentration, is typically employed. Usually, the ratio chosen represents a balance between maximizing the reaction while minimizing side reactions (often non-specific) that can impair the desired pharmacological activity of the NgR polypeptide moiety. Preferably, at least 50% of the biological activity (as demonstrated, e.g., in any of the assays described herein or known in the art) of the NgR polypeptide is retained, and most preferably nearly 100% is retained.

The polymer can be conjugated to the NgR polypeptide using conventional chemistry. For example, a polyalkylene glycol moiety can be coupled to a lysine epsilon amino group of the NgR polypeptide. Linkage to the lysine side chain can be performed with an N-hydroxysuccinimide (NES) active ester such as PEG succinimidyl succinate (SS-PEG) and succinimidyl propionate (SPA-PEG). Suitable polyalkylene glycol moieties include, e.g., carboxymethyl-NHS and norleucine-NHS, SC. These reagents are commercially available. Additional amine-reactive PEG linkers can be substituted for the succinimidyl moiety. These include, e.g., isothiocyanates, nitrophenylcarbonates (PNP), epoxides, benzotriazole carbonates, SC-PEG, tresylate, aldehyde, epoxide, carbonylimidazole and PNP carbonate. Conditions are usually optimized to maximize the selectivity and extent of reaction. Such optimization of reaction conditions is within ordinary skill in the art.

PEGylation can be carried out by any of the PEGylation reactions known in the art. See, e.g., Focus on Growth Factors, 3: 4-10, 1992 and European patent applications EP 0 154 316 and EP 0 401 384. PEGylation may be carried out using an acylation reaction or an alkylation reaction with a reactive polyethylene glycol molecule (or an analogous reactive water-soluble polymer).

PEGylation by acylation generally involves reacting an active ester derivative of polyethylene glycol. Any reactive PEG molecule can be employed in the PEGylation. PEG esterified to N-hydroxysuccinimide (NHS) is a frequently used activated PEG ester. As used herein, “acylation” includes without limitation the following types of linkages between the therapeutic protein and a water-soluble polymer such as PEG: amide, carbamate, urethane, and the like. See, e.g., Bioconjugate Chem. 5: 133-140, 1994. Reaction parameters are generally selected to avoid temperature, solvent, and pH conditions that would damage or inactivate the NgR polypeptide.

Generally, the connecting linkage is an amide and typically at least 95% of the resulting product is mono-, di- or tri-PEGylated. However, some species with higher degrees of PEGylation may be formed in amounts depending on the specific reaction conditions used. Optionally, purified PEGylated species are separated from the mixture, particularly unreacted species, by conventional purification methods, including, e.g., dialysis, salting-out, ultrafiltration, ion-exchange chromatography, gel filtration chromatography, hydrophobic exchange chromatography, and electrophoresis.

PEGylation by alkylation generally involves reacting a terminal aldehyde derivative of PEG with an NgR1 polypeptide or polypeptide fragment of the invention in the presence of a reducing agent. In addition, one can manipulate the reaction conditions to favor PEGylation substantially only at the N-terminal amino group of the NgR polypeptide, i.e. a mono-PEGylated protein. In either case of mono-PEGylation or poly-PEGylation, the PEG groups are typically attached to the protein via a —CH₂—NH— group. With particular reference to the —CH₂— group, this type of linkage is known as an “alkyl” linkage.

Derivatization via reductive alkylation to produce an N-terminally targeted mono-PEGylated product exploits differential reactivity of different types of primary amino groups (lysine versus the N-terminal) available for derivatization. The reaction is performed at a pH that allows one to take advantage of the pKa differences between the epsilon-amino groups of the lysine residues and that of the N-terminal amino group of the protein. By such selective derivatization, attachment of a water-soluble polymer that contains a reactive group, such as an aldehyde, to a protein is controlled: the conjugation with the polymer takes place predominantly at the N-terminus of the protein and no significant modification of other reactive groups, such as the lysine side chain amino groups, occurs.

The polymer molecules used in both the acylation and alkylation approaches are selected from among water-soluble polymers. The polymer selected is typically modified to have a single reactive group, such as an active ester for acylation or an aldehyde for alkylation, so that the degree of polymerization may be controlled as provided for in the present methods. An exemplary reactive PEG aldehyde is polyethylene glycol propionaldehyde, which is water stable, or mono C₁-C₁₀ alkoxy or aryloxy derivatives thereof (see, e.g., Harris et al, U.S. Pat. No. 5,252,714). The polymer may be branched or unbranched. For the acylation reactions, the polymer(s) selected typically have a single reactive ester group. For reductive alkylation, the polymer(s) selected typically have a single reactive aldehyde group. Generally, the water-soluble polymer will not be selected from naturally occurring glycosyl residues, because these are usually made more conveniently by mammalian recombinant expression systems.

Methods for preparing a PEGylated NgR polypeptides of the invention generally includes the steps of (a) reacting an NgR1 polypeptide or polypeptide fragment of the invention with polyethylene glycol (such as a reactive ester or aldehyde derivative of PEG) under conditions whereby the molecule becomes attached to one or more PEG groups, and (b) obtaining the reaction product(s). In general, the optimal reaction conditions for the acylation reactions will be determined case-by-case based on known parameters and the desired result. For example, a larger the ratio of PEG to protein, generally leads to a greater the percentage of poly-PEGylated product.

Reductive alkylation to produce a substantially homogeneous population of mono-polymer/N&R polypeptide generally includes the steps of: (a) reacting an NgR1 polypeptide or polypeptide fragment of the invention with a reactive PEG molecule under reductive alkylation conditions, at a pH suitable to permit selective modification of the N-terminal amino group of NgR; and (b) obtaining the reaction product(s).

For a substantially homogeneous population of mono-polymer/NgR polypeptide, the reductive alkylation reaction conditions are those that permit the selective attachment of the water-soluble polymer moiety to the N-terminus of an NgR1 polypeptide or polypeptide fragment of the invention. Such reaction conditions generally provide for pKa differences between the lysine side chain amino groups and the N-terminal amino group. For purposes of the present invention, the pH is generally in the range of 3-9, typically 3-6.

NgR polypeptides of the invention can include a tag, e.g., a moiety that can be subsequently released by proteolysis. Thus, the lysine moiety can be selectively modified by first reacting a His-tag modified with a low-molecular-weight linker such as Traut's reagent (Pierce Chemical Company, Rockford, Ill.) which will react with both the lysine and N-terminus, and then releasing the His tag. The polypeptide will then contain a free SH group that can be selectively modified with a PEG containing a thiol-reactive head group such as a maleimide group, a vinylsulfone group, a haloacetate group, or a free or protected SH.

Traut's reagent can be replaced with any linker that will set up a specific site for PEG attachment. For example, Traut's reagent can be replaced with SPDP, SMPT, SATA, or SATP (Pierce Chemical Company, Rockford, Ill.). Similarly one could react the protein with an amine-reactive linker that inserts a maleimide (for example SMCC, AMAS, BMPS, MBS, EMCS, SMPB, SMPH, KMUS, or GMBS), a haloacetate group (SBAP, SIA, SIAB), or a vinylsulfone group and react the resulting product with a PEG that contains a free SH.

In some embodiments, the polyalkylene glycol moiety is coupled to a cysteine group of the NgR polypeptide. Coupling can be effected using, e.g., a maleimide group, a vinylsulfone group, a haloacetate group, or a thiol group.

Optionally, the NgR polypeptide is conjugated to the polyethylene-glycol moiety through a labile bond. The labile bond can be cleaved in, e.g., biochemical hydrolysis, proteolysis, or sulfhydryl cleavage. For example, the bond can be cleaved under in vivo (physiological) conditions.

The reactions may take place by any suitable method used for reacting biologically active materials with inert polymers, generally at about pH 5-8, e.g., pH 5, 6, 7, or 8, if the reactive groups are on the alpha amino group at the N-terminus. Generally the process involves preparing an activated polymer and thereafter reacting the protein with the activated polymer to produce the soluble protein suitable for formulation.

The NgR polypeptides of the invention, in certain embodiments, are soluble polypeptides. Methods for making a polypeptide soluble or improving the solubility of a polypeptide are well known in the art.

### Polynucleotides

The present invention also includes isolated polynucleotides that encode any one of the NgR polypeptides of the present invention. The invention also includes polynucleotides that hybridize under moderately stringent or high stringency conditions to the noncoding strand, or complement, of a polynucleotide that encodes any one of the polypeptides of the invention. Stringent conditions are known to those skilled in the art and can be found in CURRENT PROTOCOLS IN MOLECULAR BIOLOGY, John Wiley & Sons, N.Y. (1989), 6.3.1-6.3.6.

The human Nogo receptor-1 polynucleotide is shown below as SEQ ID NO:1.

Full-Length Human Nogo receptor-1 is encoded by nucleotide 166 to nucleotide 1587 of SEQ ID NO: 1:

agcccagcca gagccgggcg gagcggagcg cgccgagcct
cgtcccgcgg ccgggccggg gccgggccgt agcggcggcg
cctggatgcg gacccggccg cggggagacg ggcgcccgcc
ccgaaacgac tttcagtccc cgacgcgccc cgcccaaccc
ctacgatgaa gagggcgtcc gctggaggga gccggctgct
ggcatgggtg ctgtggctgc aggcctggca ggtggcagcc
ccatgcccag gtgcctgcgt atgctacaat gagcccaagg
tgacgacaag ctgcccccag cagggcctgc aggctgtgcc
cgtgggcatc cctgctgcca gccagcgcat cttcctgcac
ggcaaccgca tctcgcatgt gccagctgcc agcttccgtg
cctgccgcaa cctcaccatc ctgtggctgc actcgaatgt
gctggcccga attgatgcgg ctgccttcac tggcctggcc
ctcctggagc agctggacct cagcgataat gcacagctcc
ggtctgtgga ccctgccaca ttccacggcc tgggccgcct
acacacgctg cacctggacc gctgcggcct gcaggagctg
ggcccggggc tgttccgcgg cctggctgcc ctgcagtacc
tctacctgca ggacaacgcg ctgcaggcac tgcctgatga
caccttccgc gacctgggca acctcacaca cctcttcctg
cacggcaacc gcatctccag cgtgcccgag cgcgccttcc
gtgggctgca cagcctcgac cgtctcctac tgcaccagaa
ccgcgtggcc catgtgcacc cgcatgcctt ccgtgacctt
ggccgcctca tgacactcta tctgtttgcc aacaatctat
cagcgctgcc cactgaggcc ctggcccccc tgcgtgccct
gcagtacctg aggctcaacg acaacccctg ggtgtgtgac
tgccgggcac gcccactctg ggcctggctg cagaagttcc
gcggctcctc ctccgaggtg ccctgcagcc tcccgcaacg
cctggctggc cgtgacctca aacgcctagc tgccaatgac
ctgcagggct gcgctgtggc caccggccct taccatccca
tctggaccgg cagggccacc gatgaggagc cgctggggct
tcccaagtgc tgccagccag atgccgctga caaggcctca
gtactggagc ctggaagacc agcttcggca ggcaatgcgc
tgaagggacg cgtgccgccc ggtgacagcc cgccgggcaa
cggctctggc ccacggcaca tcaatgactc accctttggg
actctgcctg gctctgctga gcccccgctc actgcagtgc
ggcccgaggg ctccgagcca ccagggttcc ccacctcggg
ccctcgccgg aggccaggct gttcacgcaa gaaccgcacc
cgcagccact gccgtctggg ccaggcaggc agcgggggtg
gcgggactgg tgactcagaa ggctcaggtg ccctacccag
cctcacctgc agcctcaccc ccctgggcct ggcgctggtg
ctgtggacag tgcttgggcc ctgctgaccc ccagcggaca
caagagcgtg ctcagcagcc aggtgtgtgt acatacgggg
tctctctcca cgccgccaag ccagccgggc ggccgacccg
tggggcaggc caggccaggt cctccctgat ggacgcctg

The rat Nogo receptor-1 polynucleotide is shown below as SEQ ID NO:25 and is accession number NM_{—}053613 in Genbank.

atgaagaggg cgtcctccgg aggaagccgg ctgccgacat
gggtgttatg gctacaggcc tggagggtag caacgccctg
ccctggtgcc tgtgtgtgct acaatgagcc caaggtcaca
acaagccgcc cccagcaggg cctgcaggct gtacccgctg
gcatcccagc ctccagccag agaatcttcc tgcacggcaa
ccgaatctct tacgtgccag ccgccagctt ccagtcatgc
cggaatctca ccatcctgtg gctgcactca aatgcgctgg
ccgggattga tgccgcggcc ttcactggtc tgaccctcct
ggagcaacta gatcttagtg acaatgcaca gctccgtgtc
gtggacccca ccacgttccg tggcctgggc cacctgcaca
cgctgcacct agaccgatgc ggcctgcagg agctggggcc
tggcctattc cgtgggctgg cagctctgca gtacctctac
ctacaagaca acaacctgca ggcacttccc gacaacacct
tccgagacct gggcaacctc acgcatctct ttctgcatgg
caaccgtatc cccagtgttc ctgagcacgc tttccgtggc
ttgcacagtc ttgaccgtct cctcttgcac cagaaccatg
tggctcgtgt gcacccacat gccttccggg accttggccg
actcatgacc ctctacctgt ttgccaacaa cctctccatg
ctccccgcag aggtcctagt gcccctgagg tctctgcagt
acctgcgact caatgacaac ccctgggtgt gtgactgcag
ggcacgtccg ctctgggcct ggctgcagaa gttccgaggt
tcctcatccg gggtgcccag caacctaccc caacgcctgg
caggccgtga tctgaagcgc ctggctacca gtgacttaga
gggttgtgct gtggcttcgg ggcccttccg tcccttccag
accaatcagc tcactgatga ggagctgctg ggcctcccca
agtgctgcca gccggatgct gcagacaagg cctcagtact
ggaacccggg aggccggcgt ctgttggaaa tgcactcaag
ggacgtgtgc ctcccggtga cactccacca ggcaatggct
caggcccacg gcacatcaat gactctccat ttgggacttt
gcccggctct gcagagcccc cactgactgc cctgcggcct
gggggttccg agcccccggg actgcccacc acgggccccc
gcaggaggcc aggttgttcc agaaagaacc gcacccgtag
ccactgccgt ctgggccagg caggaagtgg gagcagtgga
actggggatg cagaaggttc gggggccctg cctgccctgg
cctgcagcct tgctcctctg ggccttgcac tggtactttg
gacagtgctt gggccctgct ga

The human Nogo receptor-2 polynucleotide is shown below as SEQ ID NO:26 and is accession number BK001302 in Genbank.

atgctgcccg ggctcaggcg cctgctgcaa gctcccgcct
cggcctgcct cctgctgatg ctcctggccc tgcccctggc
ggcccccagc tgccccatgc tctgcacctg ctactcatcc
ccgcccaccg tgagctgcca ggccaacaac ttctcctctg
tgccgctgtc cctgccaccc agcactcagc gactcttcct
gcagaacaac ctcatccgca cgctgcggcc aggcaccttt
gggtccaacc tgctcaccct gtggctcttc tccaacaacc
tctccaccat ctacccgggc actttccgcc acttgcaagc
cctggaggag ctggacctcg gtgacaaccg gcacctgcgc
tcgctggagc ccgacacctt ccagggcctg gagcggctgc
agtcgctgca tttgtaccgc tgccagctca gcagcctgcc
cggcaacatc ttccgaggcc tggtcagcct gcagtacctc
tacctccagg agaacagcct gctccaccta caggatgact
tgttcgcgga cctggccaac ctgagccacc tcttcctcca
cgggaaccgc ctgcggctgc tcacagagca cgtgtttcgc
ggcctgggca gcctggaccg gctgctgctg cacgggaacc
ggctgcaggg cgtgcaccgc gcggccttcc gcggcctcag
ccgcctcacc atcctctacc tgttcaacaa cagcctggcc
tcgctgcccg gcgaggcgct cgccgacctg ccctcgctcg
agttcctgcg gctcaacgct aacccctggg cgtgcgactg
ccgcgcgcgg ccgctctggg cctggttcca gcgcgcgcgc
gtgtccagct ccgacgtgac ctgcgccacc cccccggagc
gccagggccg agacctgcgc gcgctccgcg aggccgactt
ccaggcgtgt ccgcccgcgg cacccacgcg gccgggcagc
cgcgcccgcg gcaacagctc ctccaaccac ctgtacgggg
tggccgaggc cggggcgccc ccagccgatc cctccaccct
ctaccgagat ctgcctgccg aagactcgcg ggggcgccag
ggcggggacg cgcctactga ggacgactac tgggggggct
acgggggtga ggaccagcga ggggagcaga tgtgccccgg
cgctgcctgc caggcgcccc cggactcccg aggccctgcg
ctctcggccg ggctccccag ccctctgctt tgcctcctgc
tcctggtgcc ccaccacctc tga

### Vectors

Vectors comprising nucleic acids encoding NgR polypeptides of the invention may also be used to produce polypeptide for use in the methods of the invention. The choice of vector and expression control sequences to which such nucleic acids are operably linked depends on the functional properties desired, e.g., protein expression, and the host cell to be transformed.

Expression control elements useful for regulating the expression of an operably linked coding sequence are known in the art. Examples include, but are not limited to, inducible promoters, constitutive promoters, secretion signals, and other regulatory elements. When an inducible promoter is used, it can be controlled, e.g., by a change in nutrient status, or a change in temperature, in the host cell medium.

The vector can include a prokaryotic replicon, i.e., a DNA sequence having the ability to direct autonomous replication and maintenance of the recombinant DNA molecule extra-chromosomally in a bacterial host cell. Such replicons are well known in the art. In addition, vectors that include a prokaryotic replicon may also include a gene whose expression confers a detectable marker such as a drug resistance. Examples of bacterial drug-resistance genes are those that confer resistance to ampicillin or tetracycline.

Vectors that include a prokaryotic replicon can also include a prokaryotic or bacteriophage promoter for directing expression of the coding gene sequences in a bacterial host cell. Promoter sequences compatible with bacterial hosts are typically provided in plasmid vectors containing convenient restriction sites for insertion of a DNA segment to be expressed. Examples of such plasmid vectors are pUC8, pUC9, pBR322 and pBR329 (BioRad Laboratories, Hercules, Calif.), pPL and pKK223. Any suitable prokaryotic host can be used to express a recombinant DNA molecule encoding a protein used in the methods of the invention.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. The neomycin phosphotransferase (neo) gene is an example of a selectable marker gene (Southern et al., J. Mol. Anal. Genet. 1:327-341 (1982)). Additional elements may also be needed for optimal synthesis of mRNA. These elements may include signal sequences, splice signals, as well as transcriptional promoters, enhancers, and termination signals.

In one embodiment, a proprietary expression vector of Biogen IDEC, Inc., referred to as NEOSPLA (U.S. Pat. No. 6,159,730) may be used. This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression upon transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). Additional eukaryotic cell expression vectors are known in the art and are commercially available. Typically, such vectors contain convenient restriction sites for insertion of the desired DNA segment. Exemplary vectors include pSVL and pKSV-10 (Pharmacia), pBPV-1, pml2d (International Biotechnologies), pTDT1 (ATCC 31255), retroviral expression vector pMIG and pLL3.7, adenovirus shuffle vector pDC315, and AAV vectors. Other exemplary vector systems are disclosed e.g., in U.S. Pat. No. 6,413,777.

In general, screening large numbers of transformed cells for those which express suitably high levels of the antagonist is routine experimentation which can be carried out, for example, by robotic systems.

Frequently used regulatory sequences for mammalian host cell expression include viral elements that direct high levels of protein expression in mammalian cells, such as promoters and enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (Adm1P)), polyoma and strong mammalian promoters such as native immunoglobulin and actin promoters. For further description of viral regulatory elements, and sequences thereof, see e.g., Stinski, U.S. Pat. No. 5,168,062; Bell, U.S. Pat. No. 4,510,245; and Schaffner, U.S. Pat. No. 4,968,615.

The recombinant expression vectors may carry sequences that regulate replication of the vector in host cells (e.g., origins of replication) and selectable marker genes. The selectable marker gene facilitates selection of host cells into which the vector has been introduced (see, e.g., Axel, U.S. Pat. Nos. 4,399,216; 4,634,665 and 5,179,017). For example, typically the selectable marker gene confers resistance to a drug, such as G418, hygromycin or methotrexate, on a host cell into which the vector has been introduced. Frequently used selectable marker genes include the dihydrofolate reductase (DHFR) gene (for use in dhfr-host cells with methotrexate selection/amplification) and the neo gene (for G418 selection).

Vectors encoding polypeptides or polypeptide fragments can be used for transformation of a suitable host cell. Transformation can be by any suitable method. Methods for introduction of exogenous DNA into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

Transformation of host cells can be accomplished by conventional methods suited to the vector and host cell employed. For transformation of prokaryotic host cells, electroporation and salt treatment methods can be employed (Cohen et al., Proc. Natl. Acad. Sci. USA 69:2110-14 (1972)). For transformation of vertebrate cells, electroporation, cationic lipid or salt treatment methods can be employed. See, e.g., Graham et al, Virology 52:456-467 (1973); Wigler et al., Proc. Natl. Acad. Sci. USA 76:1373-76 (1979).

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to NSO, SP2 cells, baby hamster kidney (BHK) cells, monkey kidney cells (COS), human hepatocellular carcinoma cells (e.g., Hep G2), A549 cells DG44 and DUXB11 (Chinese Hamster Ovary lines, DHFR minus), HELA (human cervical carcinoma), CV1 (monkey kidney line), COS (a derivative of CV1 with SV40 T antigen), R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte) and 293 (human kidney). Host cell lines are typically available from commercial services, the American Tissue Culture Collection or from published literature.

Expression of polypeptides from production cell lines can be enhanced using known techniques. For example, the glutamine synthetase (GS) system is commonly used for enhancing expression under certain conditions. See, e.g., European Patent Nos. 0 216 846, 0 256 055, and 0 323 997 and European Patent Application No. 89303964.4.

Eukaryotic cell expression vectors are known in the art and are commercially available. Typically, such vectors contain convenient restriction sites for insertion of the desired DNA segment. Exemplary vectors include pSVL and pKSV-10, pBPV-1, pm12d, pTDT1 (ATCC 31255), retroviral expression vector pMIG, adenovirus shuttle vector pDC315, and AAV vectors.

Eukaryotic cell expression vectors may include a selectable marker, e.g., a drug resistance gene. The neomycin phosphotransferase (neo) gene is an example of such a gene (Southern et al, J. Mol. Anal. Genet. 1:327-341 (1982)).

Frequently used regulatory sequences for mammalian host cell expression include viral elements that direct high levels of protein expression in mammalian cells, such as promoters and enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (Adm1P)), polyoma and strong mammalian promoters such as native immunoglobulin and actin promoters. For further description of viral regulatory elements, and sequences thereof, see e.g., Stinski, U.S. Pat. No. 5,168,062; Bell, U.S. Pat. No. 4,510,245; and Schaffner, U.S. Pat. No. 4,968,615.

The recombinant expression vectors may carry sequences that regulate replication of the vector in host cells (e.g., origins of replication) and selectable marker genes. The selectable marker gene facilitates selection of host cells into which the vector has been introduced (see, e.g., Axel, U.S. Pat. Nos. 4,399,216; 4,634,665 and 5,179,017). For example, typically the selectable marker gene confers resistance to a drug, such as G418, hygromycin or methotrexate, on a host cell into which the vector has been introduced. Frequently used selectable marker genes include the dihydrofolate reductase (DHFR) gene (for use in dhfr-host cells with methotrexate selection/amplification) and the neo gene (for G418 selection).

Nucleic acid molecules encoding NgR polypeptides of the invention, and vectors comprising these nucleic acid molecules, can be used for transformation of a suitable host cell. Transformation can be by any suitable method. Methods for introduction of exogenous DNA into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

Transformation of host cells can be accomplished by conventional methods suited to the vector and host cell employed. For transformation of prokaryotic host cells, electroporation and salt treatment methods can be employed (Cohen et al., Proc. Natl. Acad. Sci. USA 69:2110-14 (1972)). For transformation of vertebrate cells, electroporation, cationic lipid or salt treatment methods can be employed. See, e.g., Graham et al., Virology 52:456-467 (1973); Wigler et al., Proc. Natl. Acad. Sci. USA 76:1373-76 (1979).

### Host Cells

Host cells for expression of an NgR1 polypeptide or polypeptide fragment of the invention for use in a method of the invention may be prokaryotic or eukaryotic. Exemplary eukaryotic host cells include, but are not limited to, yeast and mammalian cells, e.g., Chinese hamster ovary (CHO) cells (ATCC Accession No. CCL61), NIH Swiss mouse embryo cells NIH-3T3 (ATCC Accession No. CRL1658), and baby hamster kidney cells (BHK). Other useful eukaryotic host cells include insect cells and plant cells. Exemplary prokaryotic host cells are E. coli and Streptomyces.

Mammalian cell lines available as hosts for expression are known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines.

Expression of polypeptides from production cell lines can be enhanced using known techniques. For example, the glutamine synthetase (GS) system is commonly used for enhancing expression under certain conditions. See, e.g., European Patent Nos. 0 216 846, 0 256 055, and 0 323 997 and European Patent Application No. 89303964.4.

### Gene Therapy

An NgR1 polypeptide or polypeptide fragment of the invention can be produced in vivo in a mammal, e.g., a human patient, using a gene-therapy approach to treatment of a nervous-system disease, disorder or injury in which antagonizing NgR-mediating signaling would be therapeutically beneficial. This involves administration of a suitable NgR polypeptide encoding nucleic acid operably linked to suitable expression control sequences. Generally, these sequences are incorporated into a viral vector. Suitable viral vectors for such gene therapy include adenoviral vectors, lentiviral vectors, baculoviral vectors, Epstein Barr viral vectors, papovaviral vectors, vaccinia viral vectors, herpes simplex viral vectors, and adeno-associated virus (AAV) vectors. The viral vector can be a replication-defective viral vector. Adenoviral vectors that have a deletion in its E1 gene or E3 gene are typically used. When an adenoviral vector is used, the vector usually does not have a selectable marker gene.

### Pharmaceutical Compositions

The NgR polypeptides, polypeptide fragments, polynucleotides, vectors and host cells of the invention may be formulated into pharmaceutical compositions for administration to mammals, including humans. The pharmaceutical compositions used in the methods of this invention comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions used in the methods of the present invention may be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. As described previously, NgR polypeptides of the invention act in the nervous system to inhibit NgR-mediated signaling. Accordingly, in the methods of the invention, the NgR polypeptides are administered in such a way that they cross the blood-brain barrier. This crossing can result from the physico-chemical properties inherent in the NgR polypeptide molecule itself, from other components in a pharmaceutical formulation, or from the use of a mechanical device such as a needle, cannula or surgical instruments to breach the blood-brain barrier. Where the NgR polypeptide is a molecule that does not inherently cross the blood-brain barrier, e.g., a fusion to a moiety that facilitates the crossing, suitable routes of administration are, e.g., intrathecal or intracranial, e.g., directly into a chronic lesion of MS. Where the NgR polypeptide is a molecule that inherently crosses the blood-brain barrier, the route of administration may be by one or more of the various routes described below.

Sterile injectable forms of the compositions used in the methods of this invention may be aqueous or oleaginous suspension. These suspensions may be formulated according to techniques known in the art using suitable dispersing or wetting agents and suspending agents. The sterile, injectable preparation may also be a sterile, injectable solution or suspension in a non-toxic parenterally acceptable diluent or solvent, for example as a suspension in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered at specific fixed or variable intervals, e.g., once a day, or on an “as needed” basis.

Certain pharmaceutical compositions used in the methods of this invention may be orally administered in an acceptable dosage form including, e.g., capsules, tablets, aqueous suspensions or solutions. Certain pharmaceutical compositions also may be administered by nasal aerosol or inhalation. Such compositions may be prepared as solutions in saline, employing benzyl alcohol or other suitable preservatives, absorption promoters to enhance bioavailability, and/or other conventional solubilizing or dispersing agents.

The amount of an NgR1 polypeptide or polypeptide fragment of the invention that may be combined with the carrier materials to produce a single dosage form will vary depending upon the host treated and the particular mode of administration. The composition may be administered as a single dose, multiple doses or over an established period of time in an infusion. Dosage regimens also may be adjusted to provide the optimum desired response (e.g., a therapeutic or prophylactic response).

The methods of the invention use a “therapeutically effective amount” or a “prophylactically effective amount” of an NgR polypeptide. Such a therapeutically or prophylactically effective amount may vary according to factors such as the disease state, age, sex, and weight of the individual. A therapeutically or prophylactically effective amount is also one in which any toxic or detrimental effects are outweighed by the therapeutically beneficial effects.

A specific dosage and treatment regimen for any particular patient will depend upon a variety of factors, including the particular NgR polypeptide used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

In the methods of the invention the NgR polypeptides are generally administered directly to the nervous system, intracerebroventricularly, or intrathecally, e.g. into a chronic lesion of MS. Compositions for administration according to the methods of the invention can be formulated so that a dosage of 0.001-10 mg/kg body weight per day of the NgR polypeptide is administered. In some embodiments of the invention, the dosage is 0.01-1.0 mg/kg body weight per day. In some embodiments, the dosage is 0.001-0.5 mg/kg body weight per day.

Supplementary active compounds also can be incorporated into the compositions used in the methods of the invention. For example, an NgR1 polypeptide or polypeptide fragment of the invention, or a fusion protein thereof, may be coformulated with and/or coadministered with one or more additional therapeutic agents.

For treatment with an NgR1 polypeptide or polypeptide fragment of the invention, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly.

In some methods, two or more NgR1 polypeptides or polypeptide fragments of are administered simultaneously, in which case the dosage of each polypeptide administered falls within the ranges indicated. Supplementary active compounds also can be incorporated into the compositions used in the methods of the invention. For example, an anti-NgR1 antibody or other NgR1 antagonist may be coformulated with and/or coadministered with one or more additional therapeutic agents.

The invention encompasses any suitable delivery method for an NgR1 polypeptide or polypeptide fragment of to a selected target tissue, including bolus injection of an aqueous solution or implantation of a controlled-release system. Use of a controlled-release implant reduces the need for repeat injections.

The NgR1 polypeptides and polypeptide fragments used in the methods of the invention may be directly infused into the brain. Various implants for direct brain infusion of compounds are known and are effective in the delivery of therapeutic compounds to human patients suffering from neurological disorders. These include chronic infusion into the brain using a pump, stereotactically implanted, temporary interstitial catheters, permanent intracranial catheter implants, and surgically implanted biodegradable implants. See, e.g., Gill et al., supra; Scharfen et al., “High Activity Iodine-125 Interstitial Implant For Gliomas,” Int. J. Radiation Oncology Biol. Phys. 24(4):583-91 (1992); Gaspar et al., “Permanent ¹²⁵I Implants for Recurrent Malignant Gliomas,” Int. J. Radiation Oncology Biol. Phys. 43(5):977-82 (1999); chapter 66, pages 577-580, Bellezza et al., “Stereotactic Interstitial Brachytherapy,” in Gildenberg et al., Textbook of Stereotactic and Functional Neurosurgery, McGraw-Hill (1998); and Brem et al, “The Safety of Interstitial Chemotherapy with BCNU-Loaded Polymer Followed by Radiation Therapy in the Treatment of Newly Diagnosed Malignant Gliomas: Phase I Trial,” J. Neuro-Oncology 26:111-23 (1995).

The compositions may also comprise an NgR1 polypeptide or polypeptide fragment of the invention dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly(2-hydroxyethyl-methacrylate), ethylene vinyl acetate (Langer et al, J. Biomed. Mater. Res. 15: 167-277 (1981); Langer, Chem. Tech. 12:98-105 (1982)) or poly-D-(−)-3hydroxybutyric acid (EP 133,988).

In some embodiments, an NgR1 polypeptide or polypeptide fragment of the invention is administered to a patient by direct infusion into an appropriate region of the brain. See, e.g., Gill et al., “Direct brain infusion of glial cell line-derived neurotrophic factor in Parkinson disease,” Nature Med. 9: 589-95 (2003). Alternative techniques are available and may be applied to administer an NgR polypeptide according to the invention. For example, stereotactic placement of a catheter or implant can be accomplished using the Riechert-Mundinger unit and the ZD (Zamorano-Dujovny) multipurpose localizing unit. A contrast-enhanced computerized tomography (CT) scan, injecting 120 ml of omnipaque, 350 mg iodine/ml, with 2 mm slice thickness can allow three-dimensional multiplanar treatment planning (STP, Fischer, Freiburg, Germany). This equipment permits planning on the basis of magnetic resonance imaging studies, merging the CT and MRI target information for clear target confirmation.

The Leksell stereotactic system (Downs Surgical, Inc., Decatur, Ga.) modified for use with a GE CT scanner (General Electric Company, Milwaukee, Wis.) as well as the Brown-Roberts-Wells (BRW) stereotactic system (Radionics, Burlington, Mass.) can be used for this purpose. Thus, on the morning of the implant, the annular base ring of the BRW stereotactic frame can be attached to the patient's skull. Serial CT sections can be obtained at 3 mm intervals though the (target tissue) region with a graphite rod localizer frame clamped to the base plate. A computerized treatment planning program can be run on a VAX 11/780 computer (Digital Equipment Corporation, Maynard, Mass.) using CT coordinates of the graphite rod images to map between CT space and BRW space.

### In Vitro Methods

The present invention also includes methods of suppressing neuronal cell growth inhibition in vitro. For example, the invention includes in vitro methods for stimulating neuronal cell growth in the presence of factors that, under normal circumstances, cause neuronal cell growth inhibition or growth cone collapse.

The methods, according to this aspect of the invention, comprise contacting a neuronal cell that expresses a Nogo receptor with an agent that causes NgR-mediated growth inhibition in the presence and absence of an isolated an NgR1 polypeptide or polypeptide fragment of the invention. As used herein, the expression “agent that causes NgR-mediated growth inhibition” means any compound that interacts with a component of the Nogo receptor signal transduction pathway (e.g., NgR or NgR interacting proteins), thereby stimulating the inhibition of neuronal cell growth or growth cone collapse. Exemplary agents that cause NgR-mediated growth inhibition include, e.g., Nogo (e.g., NogoA, Nogo-66), myelin-associated glycoprotein (AG), oligodendrocyte glycoprotein (OMgp), and fragments and derivatives thereof that inhibit the growth of cells that express the Nogo receptor. Myelin itself is another exemplary agent that causes NgR-mediated growth inhibition.

The neuronal cell used in the practice of the in vitro methods of the invention may, in certain embodiments, express an endogenous Nogo receptor. In other embodiments, the neuronal cell expresses an exogenous Nogo receptor from a vector. The neuronal cell may express both an endogenous Nogo receptor and an exogenous Nogo receptor.

The methods according to this aspect of the invention may comprise monitoring the extent of neuronal growth inhibition or growth cone collapse in the presence and/or absence of an isolated an NgR1 polypeptide or polypeptide fragment of the invention. The in vitro methods of the invention can be used to characterize the extent to which candidate NgR polypeptides are able to suppress neuronal cell growth inhibition or growth cone collapse that normally occurs in the presence of an agent that causes NgR-mediated growth inhibition. Thus, the methods of the invention are useful for identifying and characterizing the full range of NgR polypeptides having the ability to suppress neuronal cell growth inhibition. The methods according to this aspect of the invention may be performed in high throughput formats.

Other in vitro and in vivo methods for testing the ability of NgR1 polypeptides and polypeptide fragments to inhibit neurite outgrowth are described in PCT publication WO2005/016955 (incorporated herein by reference).

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Purity and Bioactivity of NgR1 Proteins

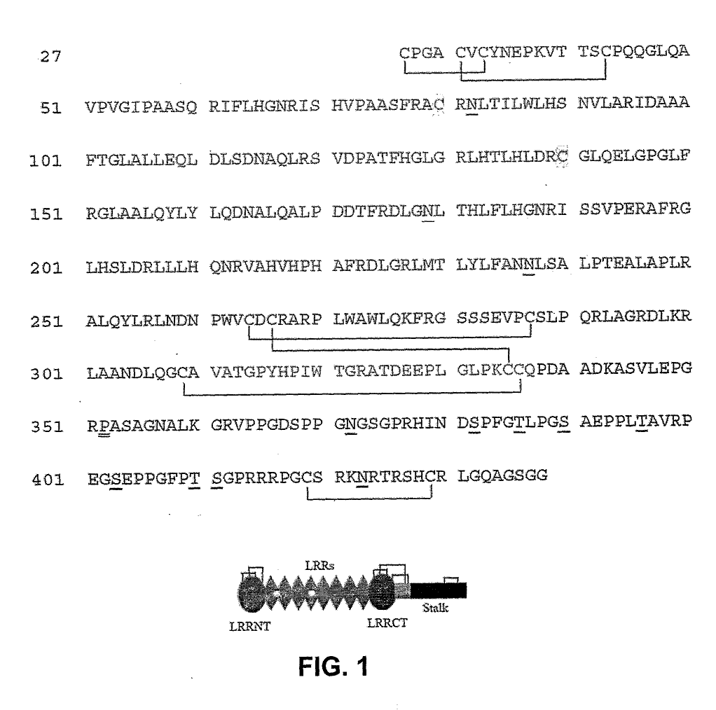

Previous deletion analyses suggest that the entire LRR region of Nogo receptor-1, including the C-terminal cap of LRR, LRRCT, is needed for ligand binding, and that the adjacent CT stalk of the Nogo receptor-1 contributes to interaction with its co-receptors (e.g., p75, TAJ, and LINGO-1). To further elucidate what regions of NgR1 were involved in the interaction with its coreceptors, various constructs of NgR1 were analyzed for their ability to bind to the coreceptors. Human NgR1, excluding the GPI domain (FL-NgR1, residues 27-438, FIG. 1 (SEQ ID NO:22)) with a flag tag at its N-terminus was expressed in CHO cells and purified as a soluble protein from the conditioned medium by sequential chromatography steps on TMAE-Fractogel (EM Merck) and Ni-NTA agarose (Qiagen). Human NgR1(310) (residues 27-310) and human NgR1(344) (residues 27-344) were expressed as histidine-tagged proteins (C-terminal tag) in insect cells and purified by sequential steps on SP-Sepharose (Amersham BioSciences) and Ni-NTA agarose. Rat NgR1(344) (residues 27-344)-rat-Fc(IgG1) and Rat NgR1(310) (residues 27-310) were expressed in CHO cells. Rat NgR1(344)-rat-Fc was purified on Protein A Sepharose (Amersham BioSciences) and Rat NgR1(310) on SP-Sepharose. Samples were analyzed for purity by SDS-PAGE on 4-20% gradient gels (NOVEX), and for aggregation by size exclusion chromatography (SEC) on a Superdex™ 200 column (Amersham Biosciences). The column was run in PBS at a flow rate of 20 mL/h and the column effluent monitored for absorbance at 280 nm.

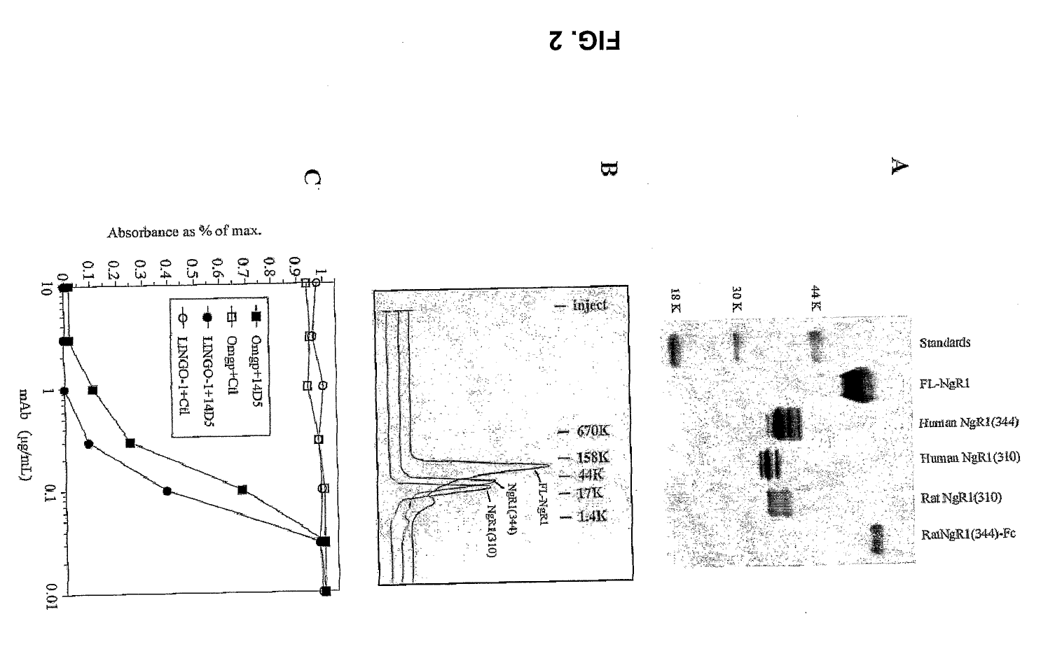

SDS-PAGE indicated that the purity of FL-NgR1 was greater than 90% with an average molecular mass of about 65 kDa (FIG. 2A). On size exclusion chromatography (SEC), the protein eluted as a single peak with a mass of about 80 kDa (FIG. 2B). FL-NgR1 was tested for binding in an ELISA assay using methods known in the art, and was found to bind LINGO-1, OMgp, Nogo-66, p75 and TAJ as well as or better than truncated versions containing the LRR region alone. See, for e.g., Shao, et al., (2005), Neuron 45, 353-359. A 10-fold higher affinity for Taj was seen using FL-NgR1 compared with the truncated versiox NgR1 (310) containing just the LRR region as described in Id. Further analysis of binding in a competition ELISA, using an anti-NgR1 antibody to block the binding of AP-OMgp and AP-Lingo-1 to NgRI, verified the activities of FL-NgR1 (FIG. 2C).

### Example 2

### Analysis of the Amino Acid Sequence of Full-Length Human NgR1Protein

The amino acid sequence of FL-NgR1 was confirmed by tryptic peptide mapping on a LC-MS system. The peptide mapping was done on protein samples with and without PNGase F treatment. First, N-linked glycans were removed from the native proteins with PNGase F. About 1 μL of PNGase F (2.5 mU/μL, Prozyme) was added to 25 μL of a solution containing about 20 μg protein; the solution was incubated at 37° C. for 24 h. Then another 1 μL of PNGase F was added and the solution was kept at room temperature for an additional 24 h. Alkylation was done under denaturing, but non-reducing conditions. About 0.3 μL of 4-vinylpyridine was added into 50 μL of the protein solution, and immediately afterwards 50 mg of guanidine hydrochloride (GuHC1) was added to the solution. The solution was incubated at room temperature in the dark for 60 min. The alkylated proteins were recovered by precipitation with 40 volumes of cooled ethanol as described in Pepinsky, R. B. (1991) Anal. Biochem. 195, 177-181. The solution was stored at −20° C. for 1 h and then centrifuged at 14000×g for 8 min at 4° C. The supernatant was discarded and the precipitate (˜20 μg/vial) was washed once with cooled ethanol.

Trypsin was chosen as the cleavage enzyme for disulfide bond linkage studies since it was expected to generate the simplest set of Cysteine-containing peptides. Digestions were performed at pH 6.5 to minimize disulfide exchange. To overcome the problem of the lower rate of hydrolysis by trypsin at pH 6.5, the proteins were treated with endo-Lys-C protease before trypsin cleavage. About 20 μg each of the alkylated proteins, deglycosylated or fully glycosylated, was digested with 5% (w/w) of endo-protease Lys-C (endo-Lys-C, Wako) in 1 M urea, 0.2 M Tris-HCl, pH 6.5, 10 mM methylamine, 1 mM CaCl₂, for 5 h at room temperature; then 5% (w/w) of trypsin (Promega) was added and the solution was incubated for an additional 10-12 h at room temperature. The final volume was 55 μL. Prior to analysis of the digests on a Liquid Chromatography/Mass Spectrometry (LC-MS) system, 55 μL of freshly prepared 8M urea was added and the solution was split into two parts: one was analyzed after reduction for 1 h at 37° C. with 40 mM DTT and the other part was directly analyzed without reduction. The reduced and non-reduced digests were analyzed on an LC-MS system composed of a HPLC (2690 Alliance Separations Module), a 2487 dual wavelength UV detector, and an LCT mass spectrometer (Waters Corp., Milford, Mass.). The HPLC was equipped with a 1.0-mm×25-cm YMC C₁₈ column (AA12SO₅₂₅₀₁WT) or a 1.0-mm×25-cm Vydac C₁₈ column (218TP51) and was eluted with a 200-min gradient (0-70% acetonitrile) in 0.03% trifluoroacetic acid at a flow rate of 0.07 mL/min at a temperature of 30° C.

The peptides were separated on a C18 reverse phase column with an on-line ESI-TOF mass spectrometer. All significant peaks were identified and accounted for 97% of the predicted NgR1 sequence (Table 1). Undetected in the peptide maps were small and hydrophilic peptides that presumably co-elute with the solvent peak. In the identified peptides, eight unpredicted sites of posttranslational modification included: hydroxylation at Proline-352 (about 75%; the peak elutes at 51.5 min in FIG. 2 and is designated T31<Hyp-352> in Table 1) and O-linked glycosylation at seven sites in Peptide T34 (residues 378-414, Table 1). The hydroxylation site was identified by tandem Mass Spectrometry (MS/MS) sequencing on the 1652.9-Da peptide (data not shown). The peak containing the tryptic glycopeptide T34 (residues 378-414) was collected, and about 0.1 μg of the peptide was dried under vacuum and resuspended in 10 μl of PBS. To remove sialic acids, an aliquot of 0.5 μl of sialidase (10 mU/μL, Boehringer Mannheim) was added, after which the solution was incubated at room temperature for 20 h. Endoprotease Glu-C (endo-Glu-C, Sequencing Grade, Roche) digestion was carried out by treating the glycopeptide with 0.05 μg of the enzyme at room temperature for 24 h. The sialidase-treated tryptic peptide T34 was analyzed on a Voyager STR mass spectrometer (Applied Biosystems, Foster City, Calif.) using DHB as a matrix. The endo-Glu-C digest of desialidated T34 was analyzed on a nano-flow LC-MS system as described above. The analysis showed that the N-linked glycosylation site, Asparagine-380, in T34 is not occupied but that all four Serine and three Threonine residues in the peptide are glycosylated to some degree, although the peptide contains, mainly, a total of 4-6 O-linked glycans (data not shown). Analytical results are consistent with predictions made using the program NetOGlyc 3.1.

TABLE 1
C-MS analysis of peptides from a tryptic digest of reduced and pyridylethylated
FL-NgF

| | Residue | Retention | Observed Mol. | Calculated Mol. |
|---|---|---|---|---|
| ¥Tryptic Peptide | Numbers | Time (min) | Mass | Mass |
| T1 | *Leu + 27-38 | 57.7 | 1395.68 | 1395.60 |
| T2 | 39-61 | 67.9 | 2307.22 | 2307.20 |
| T3 | 62-68 | 43.6 | 855.49 | 855.47 |
| T4 | 69-78 | 50.6 | 1083.59 | 1083.58 |
| PE-T5 | 79-81 | | N/D | 245.15 |
| T6 + ^{§}4Hex₅HexNAc₄Fuc | 82-95 | 96.7 | 3417.67 | 3417.58 |
| T6 | 82-95 | 108.4 | 1648.94 | 1648.94 |
| T6 (deglycosylated) | 82-95 | 111.6 | 1649.92 | 1649.95 |
| T7 | 96-119 | 147.9 | 2557.38 | 2557.34 |
| T8 | 120-131 | 57.9 | 1255.64 | 1255.63 |
| T9 | 132-139 | 50.6 | 1003.54 | 1003.56 |
| PE-T10 | 140-151 | 79.2 | 1393.81 | 1393.72 |
| T11 | 152-175 | 133.1 | 2708.52 | 2708.38 |
| T12 + ^{§}Hex₅HexNAc₄Sia₁₋₂Fuc | 176-189 | 85.4 | 3374.47 | 3374.48 |
| | | | 3665.68 | 3665.58 |
| T12 (deglycosylated) | 176-189 | 99.2 | 1606.82 | 1606.85 |
| T13 | 190-196 | 31.4 | 786.44 | 786.42 |
| T14 | 197-199 | | N/D | 393.23 |
| T15 | 200-206 | 34.9 | 796.45 | 796.42 |
| T16 | 207-213 | 41.8 | 892.55 | 892.52 |
| T17 | 214-217 | 42.0 | 1169.64 | 1169.62 |
| T18 | 224-227 | 14.9 | 460.26 | 460.25 |
| T19′ (deglycosylated) | 233-250 | 116.8 | 1911.12 | 1911.07 |
| T19 (deglycosylated) | 228-250 | 168.3 | 2532.42 | 2532.39 |
| T19 + ^{§}Hex₅HexNAc₄SiaFuc₀₋₁ | 228-250 | 146.2 | 4447.0 | 4447.8 |
| | | | 4594.0 | 4593.9 |
| T20 | 251-256 | 50.6 | 762.45 | 762.44 |
| T21 | 257-267 | 65.1 | 1333.55 | 1333.55 |
| T22 | 268-277 | 92.9 | 1267.72 | 1267.72 |
| T22′ | 270-277 | 95.4 | 1040.59 | 1040.58 |
| T23-T24 | 278-292 | 56.1 | 1648.80 | 1648.80 |
| T24 | 280-292 | 52.0 | 1345.63 | 1345.63 |
| T25 | 293-296 | 17.9 | 416.23 | 416.26 |
| T26-27 | 297-300 | 34.8 | 531.34 | 531.33 |
| T28 | 301-323 | 80.6 | 2410.18 | 2410.19 |
| T29 | 324-334 | 57.1 | 1168.61 | 1168.60 |
| T30 | 335-343 | 22.5 | 949.41 | 949.36 |
| T31 <Hyp-352> | 344-360 | 51.5 | 1652.89 | 1652.89 |
| T31 | 344-360 | 54.4 | 1636.90 | 1636.89 |
| T32-T33 (deglycosylated) | 361-377 | 36.2 | 1603.78 | 1603.80 |
| T33 (deglycosylated) | 363-377 | 34.9 | 1390.64 | 1390.67 |
| T34 + 4-6 0-linked ^{§}glycans | 378-414 + 4 | 65.8 | 5228.28 | 5228.38 |
| (HexNAcHex) | 378-414 + 5 | | 5593.41 | 5593.52 |
| | 378-414 + 6 | | 5958.57 | 5958.64 |
| | 378-414 + 7 | | 6323.63 | 6323.76 |
| T35-36 | 415-421 | 17.9 | 830.45 | 830.43 |
| T37 | 422-422 | | N/D | 146.11 |
| T38 | 423-424 | | N/D | 288.15 |
| T39 | 425-426 | | N/D | 275.16 |
| T40 | 427-430 | | N/D | 501.21 |
| T41 | 431-438 | 16.5 | 645.32 | 645.31 |
| §E1(T34) + O-linked glycans | 378-401 + 2 | N/A | 3231.49 | 3231.50 |
| [from Gluc-C treated T34] | 378-401 + 3 | | 3596.59 | 3596.62 |
| HexNAcHex | 378-401 + 4 | | 3961.60 | 3961.74 |
| §E1(T34) + O-linked glycans | 402-414 + 2 | N/A | 2014.84 | 2014.85 |
| [from Gluc-C treated T34] | 402-414 + 3 | | 2379.96 | 2379.07 |
| HexNAcHex | | | | |

¥designations denote predicted tryptic peptides from FL-NgR1 sequence where T1 is the N-terminal peptide and T41 is the C-terminal peptide
*Leu is from the Flag tag at the N-terminus of FL-NgR1.
^{§}is a fraction treated with sialidase prior to mass spectrometric analysis.

### Example 3

### Analysis of Free Cysteine and Disulfide-Linked Cysteine Residues in Human NgR1 Protein

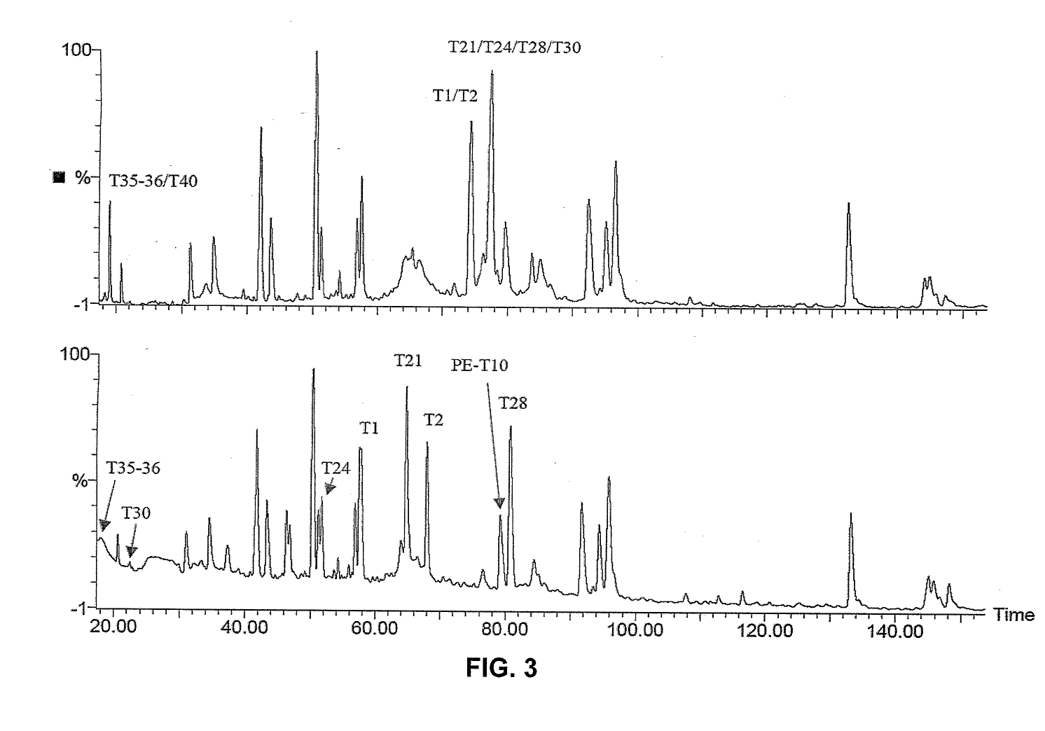

To directly assess which of the Cysteine residues in the mature structure were free, a tryptic digest of the pyridylethylated, non-reduced FL-NgR1 was analyzed on a LC-MS system after the digest had been reduced with DTT. Because the native protein was alkylated with 4-vinylpyridine prior to enzymatic cleavage, any Cysteine residues in the free thiol state should have been pyridylethylated, resulting in a 105-Da mass increase for each alkyl group. On the other hand, Cysteine residues involved in disulfide bonds should be detected as free cysteine, i.e. having a free thiol group after reduction. FL-NgR1 contains fourteen Cysteine residues—four in the LRRNT, two in the LRRs, four in the LRRCT, and four in the CT stalk. All of the predicted cysteine-containing peptides in the tryptic peptide map of the reduced digest were identified, except for those containing Cysteine-80 and Cysteine-429, which, being small, presumably eluted with the solvent peak and were not analyzed. The lower panel of FIG. 3 shows the tryptic peptide maps for the pyridylethylated FL-NgR1 after reduction. All identified peptides are listed in Table 1 with cysteine-containing peptides in bold. Analysis of these data showed that 11 of the 12 identified Cysteine residues were in the free thiol form after reduction, and that Cysteine-140 in peptide T10 (residues 140-151) was pyridylethylated. Therefore, we can infer that twelve of the Cysteine residues in native FL-NgR1 are involved in six disulfide bonds and two are unpaired. Moreover, utilizing information from the crystal structure of NgR1(310), one can predict that Cysteine-80 exists as a free thiol, since in the crystal structure it is buried in the LRR region. By inference, Cysteine-429 in the CT stalk region, not present in the crystal structure, must be involved in disulfide bond formation.

### Example 4

### Analysis of Disulfide Linkages in FL-NgR1 Protein

Disulfide structures within NgR1 were determined by analyzing peptide maps of non-reduced digests. Based on the disulfide structure seen in the crystal structure of NgR1(310) as described in He, et al, (2003) Neuron, 38, 177-185 and Barton, et al., (2003) EMBO J. 22, 3291-3302, the non-reduced digest should contain two groups of disulfide-linked peptides, one from the LRRNT region and the other from the LRRCT region. In fact, analysis of the peptide map of the non-reduced digest did reveal a group of disulfide-linked peptides (T1/T2) from the LRRNT region eluting at 74.3 min (FIG. 3, upper panel). Mass spectrometric analysis of the peak showed that it contains two peptides, T1 (residues 27-38) and T2 (residues 39-61), linked by two disulfide bonds (observed mass, 3698.77 Da; calculated mass, 3698.77 Da; Table 2). The peak containing the T1 and T2 peptides disappeared when the digest was reduced with DTT and, concomitantly, on the reduced map, two new peaks corresponding to the individual peptides, T1 and T2 (FIG. 3, lower panel) were observed. Peptide T1 contains three cysteines. Due to the lack of a protease that can cleave between Cysteine-27 and Cysteine-29, and Cysteine-29 and Cysteine-33, the exact disulfide linkages in T1/T2 had to be determined by partial reduction with Tris(2-carboxyethyl) phosphine hydrochloride (TCEP, Pierce) and alkylation with N-ethylmaleimide (NEM, Pierce) followed by LC-MS/MS analysis. To accomplish this, the disulfide-linked tryptic peptides were partially reduced using TCEP, Pierce in 0.1 M citrate buffer, pH 3, containing 6 M guanidine HCl as described in Burns, et al., J. Org. Chem. 1991, 56, 2648-2650. Various amounts of TCEP were added to the solution to find optimal conditions. The optimal amounts of TCEP were found to be 5 nmol for 20 pmol of the disulfide-linked peptides in the LRRNT region, and 5 nmol for 10 pmol of the disulfide-linked peptides in the LRRCT and stalk regions. The total volume of the solution was 2.5 μl. The reduction was carried out at 37° C. for 15 min and was stopped by alkylating the partially reduced peptides with an excess of NEM in 0.4 M citrate buffer, pH 4.5 containing 6 M guanidine HCl. The final concentration of NEM in the solution (5 p1) was 10 mM; the solution was kept at 37° C. for 1 h. The partially reduced and NEM-alkylated peptides were analyzed on a nano-flow LC-MS/MS system as described above, either directly or after further fractionation on a 2690 Alliance Separations Module with a 1.0-mm×15-cm Atlantic dC₁₋₈ column (186001283, Waters Corp.). A 70-min gradient (5-70% acetonitrile) in 0.1% trifluoroacetic acid at a flow rate of 0.07 mL/min was used, at 30° C., for fractionation. Components in peaks on the peptide maps were identified using MassLynx 4.0 software (Waters Corp.). MS/MS spectra were acquired using the data dependent acquisition function (DDA) on a nano-flow LC-MS/MS system as described above. Ramped collision energy 21-40 ev was used for MS/MS experiments and MS/MS spectra were collected in the m/z range 50-1800, with sampling every 0.5 sec, 0.05 sec separation between consecutive spectra. The MS or MS/MS spectra acquired from the Q-TOF Premier were deconvoluted by the MaxEnt 3 program. Peptides linked by disulfide bonds were further identified by comparing the map of the non-reduced digest with the map of the corresponding reduced sample.

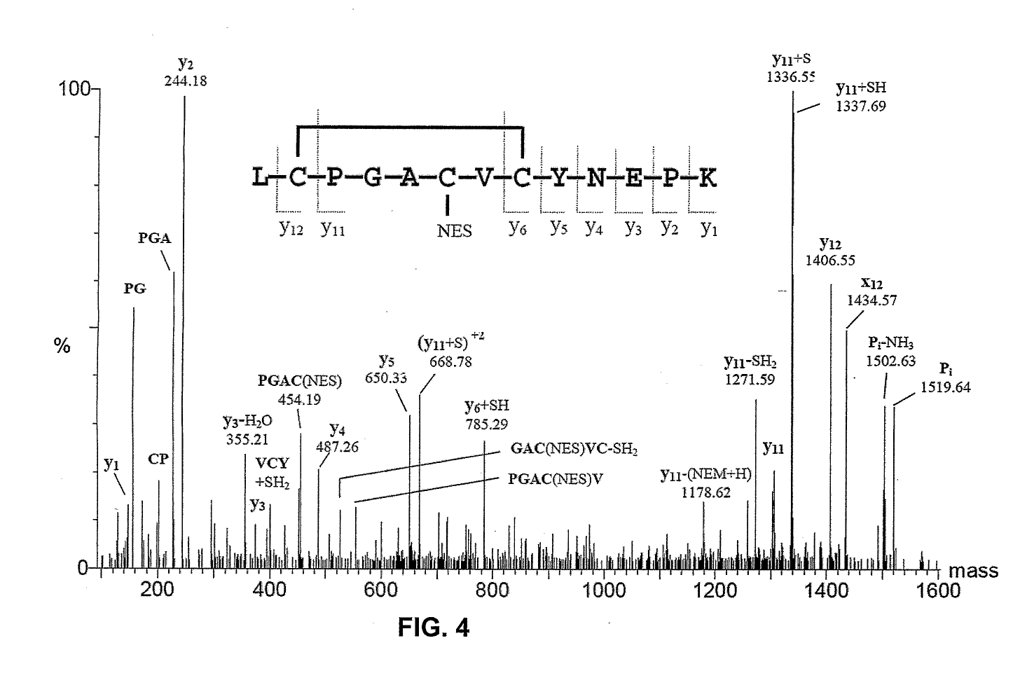

From the NgRI(310) crystal structure as described in He, et al., (2003) Neuron, 38, 177-185 and Barton, et al., (2003) EMBO J. 22, 3291-3302, we can infer that T1 will have an intra-peptide disulfide bond and is linked to T2 by an inter-peptide disulfide bond. Mass spectrometric analysis of the products of the partial reduction and alkylation, after separation on a C₁₈ column, detected the following predicted partially reduced, NEM-alkylated peptides: T1 containing one disulfide bond and one N-ethylsuccinimidyl (NES) group (observed MH⁺=1519.64, calculated MH⁺=1519.64), T2 with one NES group (observed MH⁺=2433.26, calculated MH⁺=2433.25), and T1/T2 containing one inter-peptide disulfide bond and two NES groups (observed MH⁺=3951.90, calculated MH⁺=3951.89 Da). The MS/MS spectrum for T1 containing one disulfide bond and one NES group, shown in FIG. 4, indicates that the NES group is on Cysteine-29 (internal fragment ions, PGAC(NES) and PGAC(NES)V, y₁₁ related ions, FIG. 4), which means that Cysteine-33 is linked to Cysteine-27 by an intra-peptide disulfide bond, and that Cysteine-29 is linked to Cysteine-43 in T2 by an inter-peptide disulfide bond. MS/MS sequencing results for T1/T2 containing one inter-peptide disulfide bond and two NES groups are consistent with this conclusion, since analysis showed that the two NES groups were at Cysteine-27 and Cysteine-33 (data not shown).

The crystal structure of the LRRCT region of NgR1(310) as described in He, et al., (2003) Neuron, 38, 177-185 and Barton, et al., (2003) EMBO J. 22, 3291-3302) revealed disulfide linkages of Cysteine-264 to Cysteine-287 and Cysteine-266 to Cysteine-309. Therefore, the four Cysteine residues in the LRRCT region should be contained in three tryptic peptides—T21 (residues 257-267), T24 (residues 280-292), and T28 (residues 301-323) linked together by two inter-peptide disulfide bonds (the calculated mass for this cluster should be 5088.68 Da). The three individual peptides, T21, T24, and T28 (Lower panel of FIG. 3 and Table 1), were easily identified in the map of the reduced digest, but no significant peak corresponding to this peptide cluster, T21/T24/T28 was found in the map of the non-reduced digest. Instead, a prominent peak with mass of 6032.62 Da occurred which corresponds to a four-peptide cluster containing T21, T24, T28, and T30 (residues 335-343) linked by three disulfide bonds (calculated mass=6032.68 Da; upper panel of FIG. 3, and Table 2). Since peptides T21 and T30 each contain two Cysteine residues, one Cysteine in peptide T21 must form a disulfide bond with one in peptide T30, although the exact linkages could not be determined. The tryptic peptide mapping analysis also showed that the peak at 19.0 min contains the other two Cysteine-containing peptides in the CT stalk region, and that they are linked by a disulfide bond between Cysteine-419 and Cysteine-429 (Table 2 and FIG. 3, upper panel).

To determine disulfide linkages in peptide T21/T24/T28/T30 complex, the peak containing the disulfide-linked peptides in the LRRCT and stalk region on the tryptic peptide map was collected, dried under vacuum, and resuspended in 10 μl of 0.1 M Tris-HCl, pH 6.5, 1 mM MgCl₂. About 0.02 μg of endo-protease Asp-N (endo-Asp-N, Sequencing Grade, Roche) was added to 0.6 μg of the peptides, after which the solution was incubated at room temperature for 6 h. The digest was analyzed on a nano-flow LC-MS system composed of a nano-flow HPLC (NanoAcquity, Waters Corp., Milford, Mass.) and a Q-TOF Premier mass spectrometer (Waters Corp., Milford, Mass.). A 0.10-mm×10-cm Atlantic dC₁₈ column (186002831, Waters Corp.) was used for the separation with a 50-min gradient (0-70% acetonitrile) in 0.1% formic acid at a flow rate of 400 mL/min. The temperature was 35° C.

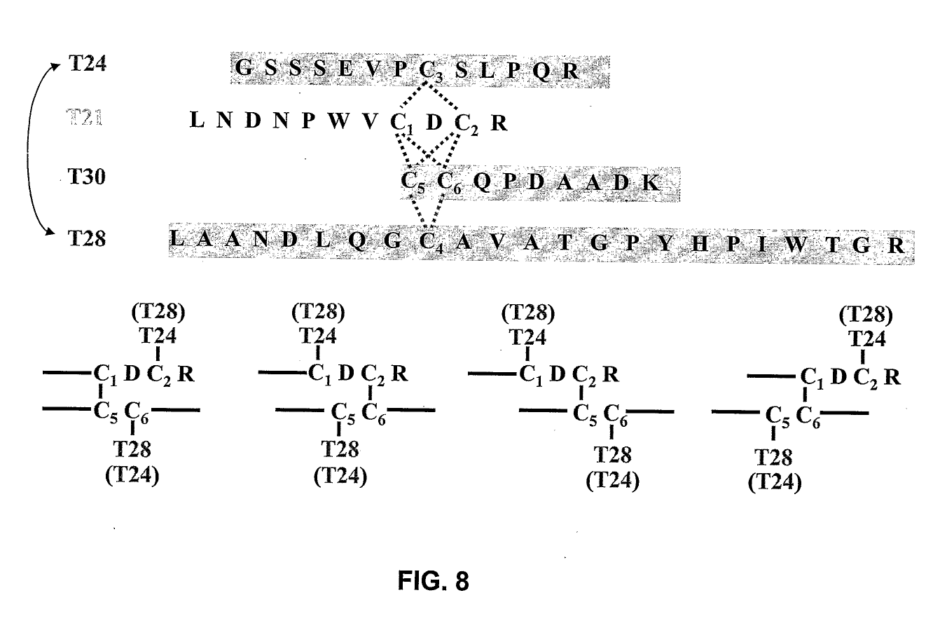

Since peptides T21 and T30 each contain two Cys residues, one Cys in peptide T21 must form a disulfide bond with one in peptide T30 (FIG. 8). There are eight possible disulfide structures for peptide T21/T24/T28/T30 cluster (FIG. 8).

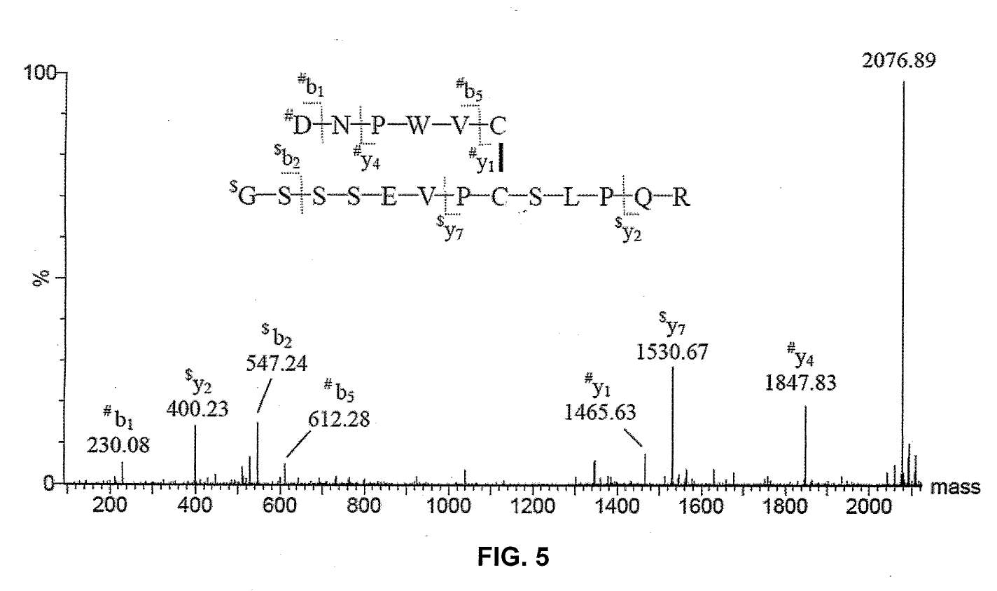

Two significant peaks were detected by mass spectrometric analysis in the non-reduced digest (data not shown). Detected MH⁺ 2076.89 (FIG. 5) in the second peak matches the calculated MH⁺ 2076.91 for peptide T21 and peptide T24 linked by a disulfide bond between Cysteine-264 and Cysteine-287, as seen in the crystal structure of NgR1(310). The identity of this fragment was confirmed by the observation of in-source fragmentation ions (FIG. 5). The observed MH⁺ 2879.50 Da in the other peak matches the calculated MH⁺ 2879.25 Da for the group of three peptides, residues 265-267 (derived from T21), residues 305-323 (derived from T28), and residues 335-338 (derived from T30), linked by two inter-peptide disulfide bonds, which indicated that Cysteine-266 and Cysteine-309 in the LRRCT region form disulfide bonds with Cysteine-335 and Cysteine-336 in the CT stalk region (data not shown). Determination of the exact disulfide pairings, Cysteine-266 and Cysteine-309 with Cysteine-335 and Cysteine-336, in this case, was complicated by the fact that no reagents exist that can cleave the backbone between Cysteine-335 and Cysteine-336.

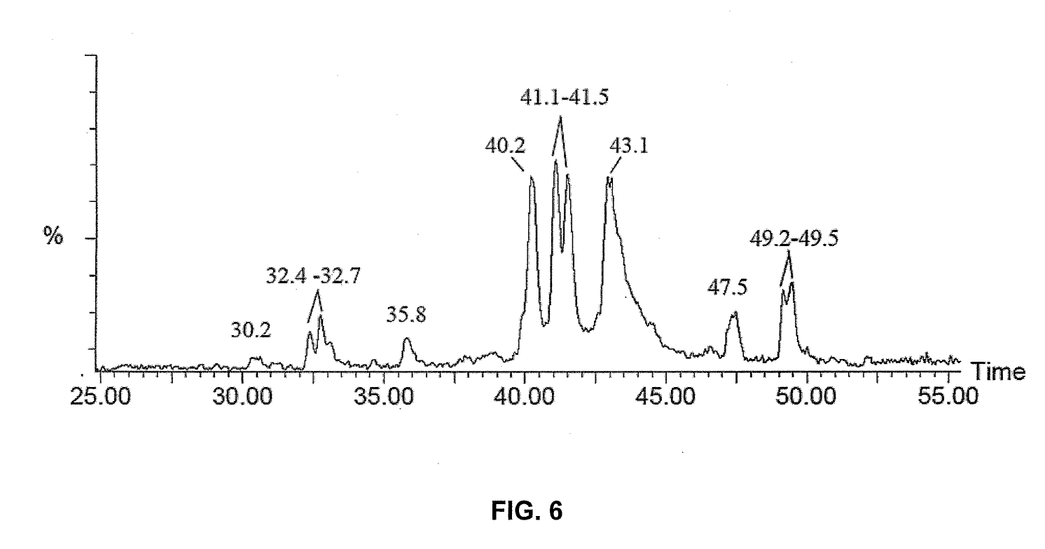

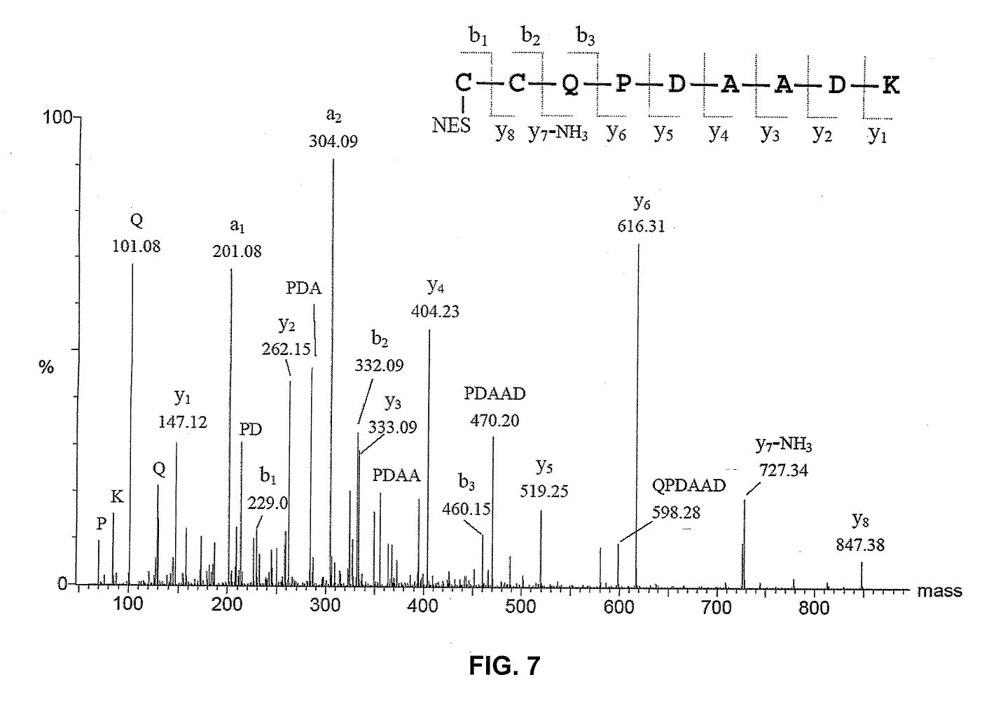

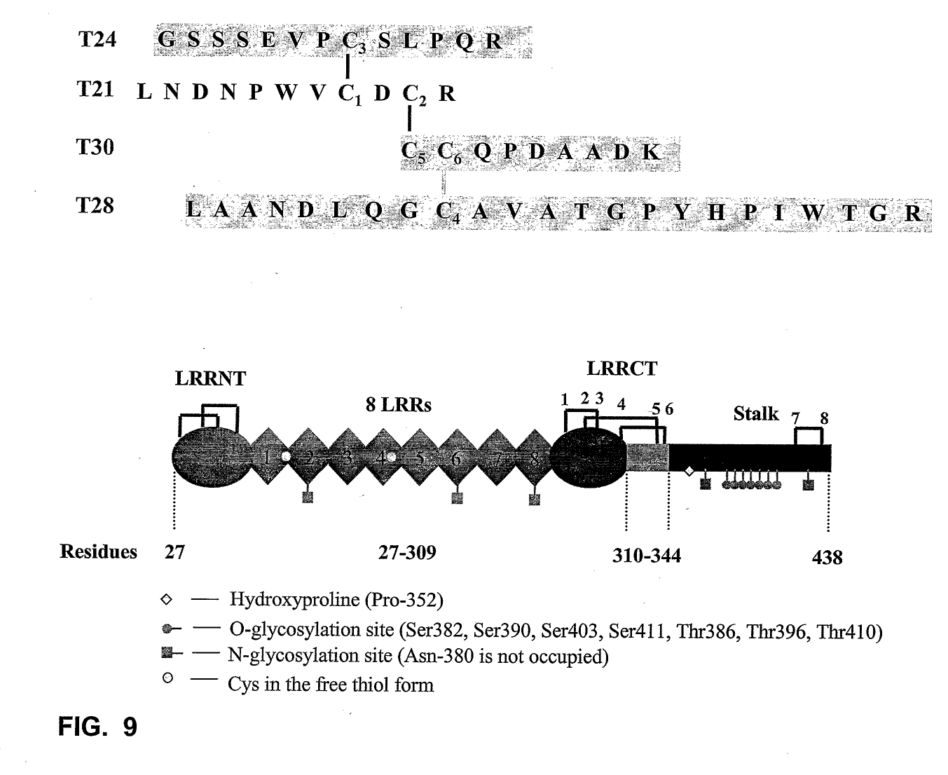

The disulfide pairing arrangement in the T21/T24/T28/T30 complex was further elucidated by subjecting it to partial reduction with TCEP followed by alkylation with NEM and analysis by nano-LC-MS. FIG. 6 shows the nano-flow LC-MS results (TIC) and Table 3 lists the identities of the components in the peaks. The doublet peaks seen for certain peptides are due to stereoisomers generated by NEM alkylation. The MS/MS spectra are the same for individual peaks in each doublet (data not shown). The doublet peak containing T28/T30 with a disulfide bond and an NES group was collected from a fractionation run on a 1-mm×150 mm column, and further analyzed on a nano-LCMS/MS system after it had been fully reduced with DTT. FIG. 7 shows the MS/MS spectrum of the peptide T30 containing a NES group. Both b₁ and y₈ ions, detected by MS/MS sequencing, show that the NES group is at Cysteine-356, not Cysteine-366, because the observed m/z is 229.08 for b, and 847.38 for y₈ (the calculated m/z value is 229.06 for b, and 847.36 for y₈, if Cysteine-335 is alkylated with NEM; the calculated m/z is 104.10 for b₁ and 972.46 for y₈, if Cysteine-336 is alkylated with NEM), which indicates that Cysteine-336 forms a disulfide bond with Cysteine-309. Consequently, then, Cysteine-335 must be linked to Cysteine-266. Experimentally determined disulfide linkages in the T21/T24/T28/T30 complex are shown in FIG. 9. Our analysis of the disulfide structure in the LRRCT domain of FL-NgR1 not only demonstrates that the predicted disulfide structure for the LRRCT of NgR1 is incorrect, but also identifies an alternative cysteine pairing structure. While not being bound by theory, it is believed that the Cys-266 to Cys-309 linkage seen in NgR1(310) is an artifact created by the truncation.

TABLE 2
Disulfide-linked peptides detected in a tryptic peptide map of the non-reduced
digest of pyridylethylated FL-NgR1

| | Residue | Retention | Observed Mol. | Calculated |
|---|---|---|---|---|
| ¥Tryptic Peptide | Numbers | Time (min) | Mass | Mol. |
| T1/T2 | *Leu + 27-38 | 74.3 | 3698.77 | 3698.77 |
| with 2 disulfide bonds | 39-61 | | | |
| T21/T24/T28/T30 | 257-267 (C1, C2) | 77.3 | 6032.62 | 6032.68 |
| with 3 disulfide bonds | 280-292 (C3) | | | |
| | 301-323 (C4) | | | |
| | 335-343 (C5, C6) | | | |
| T35-T36/T40 | 415-421 (C7) | 19.0 | 1329.62 | 1329.62 |
| with 1 disulfide bond | 427-430 (C8) | | | |

TABLE 3
LC-MS analysis of components from the partially reduced,
NEM-alkylated peptide cluster T21/T24/T28/T30

| | Retention | Observed | Calculated |
|---|---|---|---|
| ¥ Tryptic Peptide | Time (min) | Mol. Mass | Mol. Mass |
| T30 + 2 NES | 30.4 | 1199.58 | 1199.46 |
| T24 + 1 NES | 32.4-32.8 | 1470.70 | 1470.68 |
| T21/T24/T30 + 1 NES | 35.8 | 3749.56 | 3749.56 |
| T21/T24 + 1 NES | 40.25 | 2802.21 | 2802.22 |
| T28/T30 + 1 NES | 41.1-41.5 | 3482.57 | 3482.58 |
| T21/T24/T28/T30 | 43.0 | 6032.62 | 6032.68 |
| T21 + 2 NES | 47.4 | 1583.66 | 1583.65 |
| T28 + 1 NES | 49.2-49.5 | 2535.26 | 2535.23 |

### Example 5

Analysis of the Disulfide Structures of NgR1 and NgR2 Proteins Made from Different Constructs

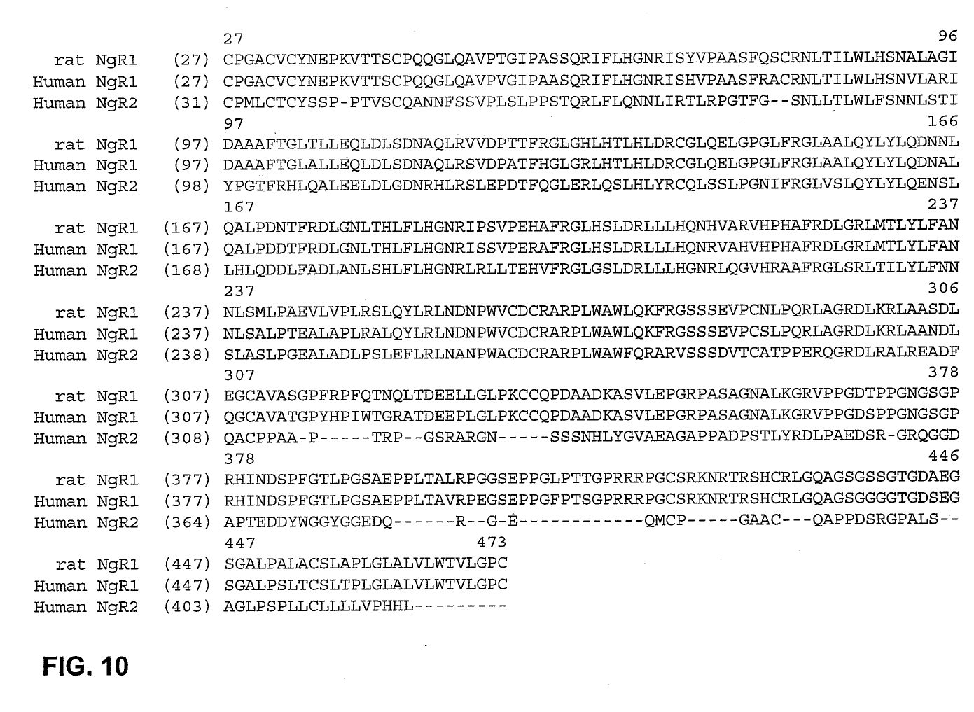

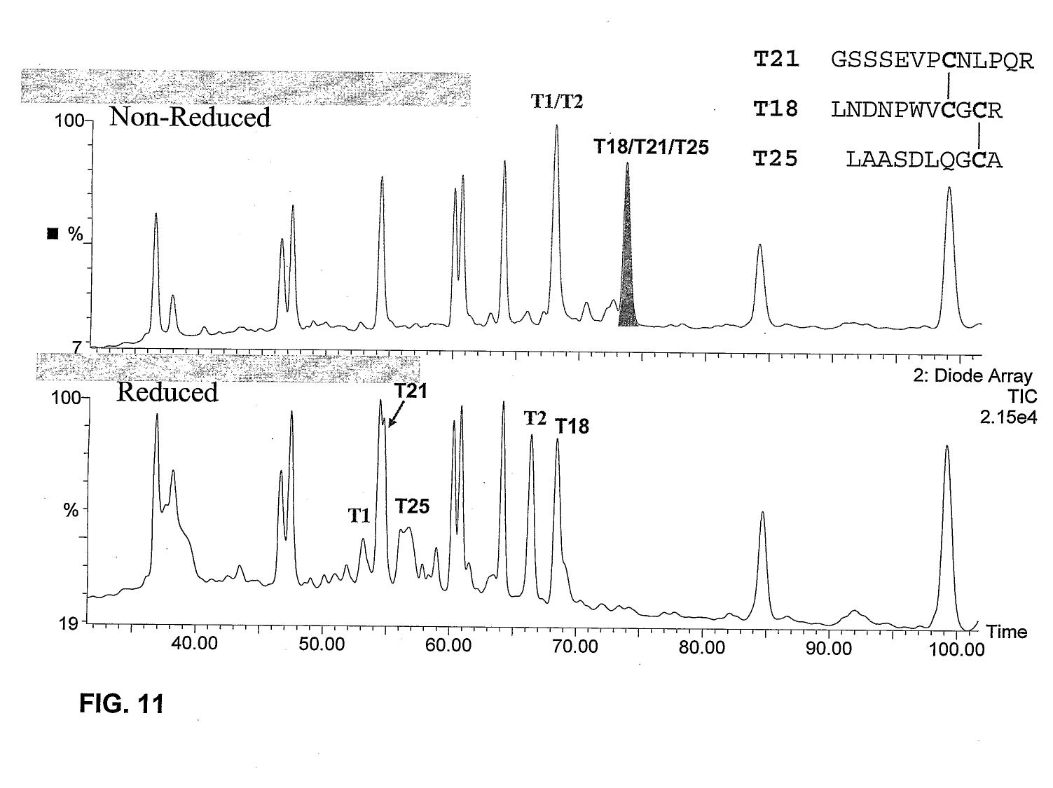

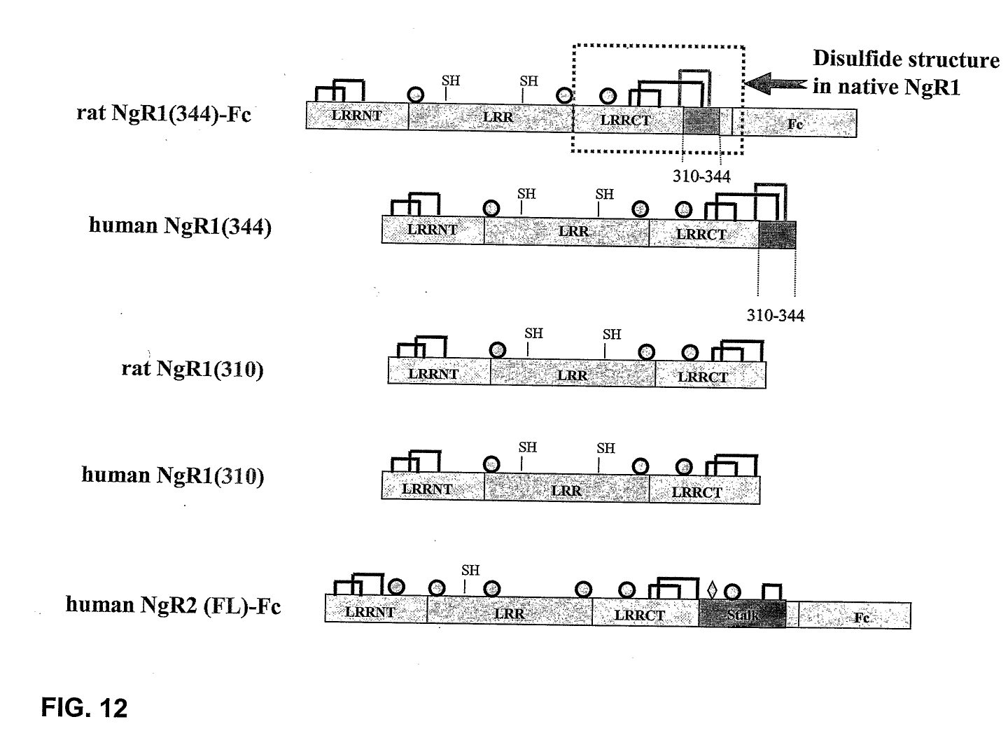

Disulfide structures in human NgR2(FL)-Fc, human NgR1(310) protein, human NgR1 (344) protein, rat NgR1 (310) protein, and rat NgR1(344)-ratFc(IgG1) fusion protein [ratNgR1(344)-Fc] were also analyzed by tryptic peptide mapping. The alignment of the sequences is shown in FIG. 10. FIG. 11 shows the tryptic peptide maps for rat NgR1(310) as an example. The results are summarized in Table 4 and FIG. 12. These analyses showed that disulfide structures in human NgR2(FL)-Fc, rat NgR1(310) and human NgR1(310) proteins which lack the two Cysteine residues, Cysteine-335 and Cysteine-336, in the CT stalk region are the same as seen in the crystal structure of human NgR1(310), and that the disulfide structures in the rat NgR1(344)-Fc and human NgR1(344) proteins which do have the two Cysteine residues in the CT stalk region are the same as seen in FL-NgR1. Mass spectrometric analysis showed the two Cys residues in the CT-stalk of NgR2(FL)-Fc are linked by a disulfide bond as seen in NgR1. Analysis also identified an O-linked glycosylation site, Thr-313, in the LRRCT of NgR2(FL)-Fc. Glycosylation site occupancy is about 35%.

TABLE 4
Summary of mass spectrometric analyses for disulfide structures in NgR1
proteins made from different constructs

| Disulfide | | | | | |
|---|---|---|---|---|---|
| Linked | Observed Mot. Mass | | | | Calculated Mol. |
| Peptides | NgR1(310) | NgR1(344) | ratNgR1(310) | ratNgR1(344)-Fc | Mass |
| T1/T2 with 2 | 3672.63 | 3672.76 | 3603.67 | 3603.64 | 3672.72 (human) |
| disulfide bonds | | | | | 3603.65 (rat) |
| T21/T24/ | 3892.45 | N/D | 3591.69 | N/D | 3892.73 (human) |
| T28 with 2 | | | | | 3591.60 (rat) |
| disulfide bonds | | | | | |
| T21/T24/ | N/A | 6036.21 | N/A | 7240.51 | 6036.72 (human) |
| T28/T30 with 3 | | | | | 7241.06 (rat) |
| disulfide bonds | | | | | |

### Example 6

### Neurite Outgrowth Assay

The effect of soluble Nogo receptor polypeptides and polypeptide fragments of the invention on neurite outgrowth is tested by performing experiments with cells grown in the presence and absence of laminin. Neuronal cell growth in media without laminin is poor and models neuronal stress conditions.

Dorsal root ganglions (DRG's) are dissected from post-natal day 6-7 rat pups (P6-7), dissociated into single cells and plated on 96-well plates pre-coated with 0.1 mg/ml poly-D-lysine (Sigma®). In some wells 2 μg/ml laminin is added for 2-3 hours and rinsed before the cells are plated. After an 18-20 h incubation the plates are fixed with 4% para-formaldehyde, stained with rabbit anti-Beta-III-tubulin antibody diluted 1:500 (Covance®) and anti-HuC/D diluted 1:100 (Molecular Probes), and fluorescent secondary antibodies (Molecular Probes) are added at 1:200 dilution.

The ArrayScan® II (Cellomics®) maybe used to capture 5× digital images and to quantify neurite outgrowth as average neurite outgrowth/neuron per well, by using the Neurite outgrowth application. Sufficient images are analyzed to allow statistical analysis of the results.

In some experiments, a sub-clone of PC12 cells (Neuroscreen) is used (Cellomics). The Neuroscreen cells are pre-differentiated for 7 days with 200 ng/ml NGF, detached and replated on 96-well plates pre-coated with poly-D-lysine. In some wells 5 μg/ml laminin is added for 2-3 hours and rinsed before the cells are plated. After 2 days incubation the plates are fixed with 4% para-formaldehyde, stained with rabbit anti-Beta-III-tubulin antibody diluted 1:500 (Covanceg) and Hoechst (nuclear stain). The ArrayScan® II is used to quantify neurite outgrowth as in the DRG cells as described above.

NgR1 polypeptides and polypeptide fragments of the invention, e.g., NgR1 (309-344) polypeptide fragment, are added in solution to P6-7 DRG neurons and to differentiated Neuroscreen™ cells at the time of plating.

The effect of the NgR1 polypeptides or polypeptide fragments on neurite outgrowth is assessed.

### Example 7

### Neurite Outgrowth Assay

Lab-Tek® culture slides (4 wells) are coated with 0.1 mg/ml poly-D-lysine (Sigma). CNS myelin alone or mixed with a NgR1 polypeptide or polypeptide fragment of the invention, e.g., NgR1 (309-344) polypeptide fragment, are separately spotted as 3 μl drops. Fluorescent microspheres (Polysciences) are added to the myelin/PBS to allow later identification of the drops (Grandpre et al., Nature 403, (2000)). Lab-Tek® slides are then rinsed and coated with 10 μg/ml laminin (Gibco™).

Dorsal root ganglions (DRG's) from P3-4 Sprague Dawley rat pups are dissociated with 1 mg/ml collagenase type 1 (Worthington), triturated with fire-polished Pasteur pipettes pre-plated to enrich in neuronal cells and finally plated at 23,000 cells/well on the pre-coated Labtek culture slides. The culture medium is, for example, F12 containing 5% heat inactivated donor horse serum, 5% heat inactivated fetal bovine serum and 50 ng/ml mNGF and incubated at 37° C. and 5% C02 for 6 hours.

Slides are fixed for 20 minutes with 4% paraformaldehyde containing 20% sucrose and stained for the neuronal marker anti beta-III-tubulin (Covance TUJ1) diluted 1:500. As secondary antibody anti-mouse Alexa Fluor® 594 (Molecular Probes) is diluted 1:300 and slides are coverslipped with Gel/Mount™ (Bimeda™). Sufficient 5× digital images are acquired with OpenLab software and analyzed by using the MetaMorph® software for quantification of neurite outgrowth.

The ability of the NgR1 polypeptide or polypeptide fragments to protect DRG neurons from myelin-mediated inhibition of neurite outgrowth is assessed.

1. An isolated polypeptide fragment of 40 residues or less, comprising an amino acid sequence identical to amino acids 309 to 344 of SEQ ID NO:2, except for up to three amino acid substitutions.

2. The polypeptide fragment of claim 1, wherein at least one of said amino acid substitutions is made at a cysteine residue selected from the group consisting of C309, C335, and C336.

3. (canceled)

4. (canceled)

5. (canceled)

6. The polypeptide fragment of claim 2, wherein said cysteine residue is substituted with a different amino acid selected from the group consisting of: alanine, serine, or threonine.

7. The polypeptide fragment of claim 6, wherein said different amino acid is alanine.

8. The polypeptide fragment of claim 1, which is cyclic.

9. (canceled)

10. (canceled)

11. (canceled)

12. (canceled)

13. (canceled)

14. The polypeptide fragment of claim 1, fused to a heterologous polypeptide.

15. The polypeptide fragment of claim 14, wherein said heterologous polypeptide is serum albumin.

16. The polypeptide fragment of claim 14, wherein said heterologous polypeptide is an Fc region.

17. The polypeptide fragment of claim 14, wherein said heterologous polypeptide is a signal peptide.

18. The polypeptide fragment of claim 14, wherein said heterologous polypeptide is a polypeptide tag.

19. (canceled)

20. (canceled)

21. The polypeptide fragment of claim 1, wherein said polypeptide is attached to one or more polyalkylene glycol moieties.

22. (canceled)

23. (canceled)

24. An isolated polynucleotide comprising a nucleotide sequence that encodes a polypeptide fragment of claim 1.

25. (canceled)

26. (canceled)

27. A vector comprising the polynucleotide of claim 24.

28. A host cell comprising the vector of claim 27.

29. A pharmaceutical composition comprising the polypeptide fragment of claim 1 and a pharmaceutically acceptable carrier.

30. A pharmaceutical composition comprising the polynucleotide of claim 24 and a pharmaceutically acceptable carrier.

31. A pharmaceutical composition comprising the vector of claim 27 and a pharmaceutically acceptable carrier.

32. A method of promoting neurite outgrowth comprising contacting a neuron with
the polypeptide fragment of claim 1;
wherein said polypeptide fragment inhibits Nogo receptor 1-mediated neurite outgrowth inhibition.

33. (canceled)

34. (canceled)

35. A method of inhibiting signal transduction by the NgR1 signaling complex, comprising contacting a neuron with an effective amount of
the polypeptide fragment of claim 1;
wherein said polypeptide fragment inhibits signal transduction by the NgR1 signaling complex.

36. (canceled)

37. (canceled)

38. A method of treating a central nervous system (CNS) disease, disorder, or injury in a mammal, comprising administering to a mammal in need of treatment an effective amount of
the polypeptide fragment of claim 1;
wherein said polypeptide fragment inhibits Nogo receptor 1-mediated neurite outgrowth inhibition.

39. (canceled)
